(19) United States
(12) Patent Application Publication
(10) Pub. No.: US 2026/0062453 A1
(43) Pub. Date: Mar. 5, 2026

(54) DEGRADATION TARGETING AGENTS AND USES THEREOF

(71) Applicant: Wisconsin Alumni Research Foundation, Madison, WI (US)

(72) Inventors: Gaelen Hess, Madison, WI (US); Grace Boyum, Madison, WI (US)

(73) Assignee: Wisconsin Alumni Research Foundation, Madison, WI (US)

(21) Appl. No.: 19/285,456

(22) Filed: Jul. 30, 2025

Related U.S. Application Data
(60) Provisional application No. 63/676,997, filed on Jul. 30, 2024.
(60) Provisional application No. 63/786,361, filed on Apr. 10, 2025.

(51) Int. Cl. C07K 14/415; C12N 15/115

(52) U.S. Cl. CPC C07K 14/415; C12N 15/115; C07K 2319/09; C07K 2319/80; C12N 2310/16

(57) ABSTRACT

Degradation targeting agents and uses thereof, such as for degrading proteins. The degradation targeting agents include a ligand moiety and a degradation targeting moiety. The degradation targeting moiety can include a degradation targeting peptide sequence that includes a C3HC4-type zinc-finger domain, such as a C3HC4-type zinc-finger domain from proteins such as helicase-like transcription factor (HLTF) and RO52. The ligand moiety can be a moiety that recruits the degradation targeting moiety to target molecules by binding to the target molecules. The degradation targeting agents can be used in methods of degrading target molecules by contacting the target molecules with the degradation targeting agents to induce degradation of the target molecules.

### CROSS-REFERENCE TO RELATED APPLICATIONS

Priority is hereby claimed to U.S. Provisional Application 63/676,997, filed Jul. 30, 2024, and U.S. Provisional Application 63/786,361, filed Apr. 10, 2025, which are incorporated herein by reference in their entireties.

### STATEMENT REGARDING FEDERALLY SPONSORED RESEARCH

This invention was made with government support under GM150462 awarded by the National Institutes of Health. The government has certain rights in the invention.

### SEQUENCE LISTING

The instant application contains a Sequence Listing which has been submitted in XML format and is hereby incorporated by reference in its entirety. The XML copy, created on Jul. 25, 2025, is named USPTO-250725-09824635-P250015US03-SEQ_LIST and is 61,757 bytes in size.

### FIELD OF THE INVENTION

The invention is directed to degradation targeting agents and uses thereof, such as for degrading proteins and other target molecules.

### BACKGROUND

Targeted degradation is a therapeutic strategy for disposing of pathogenic proteins and other target molecules. Exemplary agents that target molecules such as proteins for degradation are known as PROTACs (proteolysis targeting chimera), which utilize the proteasomal pathway, and LYTACs (lysosome targeting chimera) or AUTACs (autophagy targeting chimera), which utilize the lysosomal pathway, among others (Zhao L, Zhao J, Zhong K, Tong A, Jia D. Targeted protein degradation: mechanisms, strategies and application. Signal Transduct Target Ther. 2022 Apr. 4; 7 (1): 113). Further agents that target molecules such as proteins for degradation are needed.

### SUMMARY OF THE INVENTION

One aspect of the invention is directed to degradation targeting agents. The degradation targeting agents of the invention can comprise a ligand moiety and a degradation targeting moiety.

The degradation targeting moiety comprises a degradation targeting peptide sequence.

In some versions, the degradation targeting peptide sequence comprises a C3HC4-type zinc-finger domain. In some versions, the degradation targeting peptide sequence is at least 80%, at least 85%, at least 90%, at least 95%, at least 96%, at least 97%, at least 98%, at least 99%, or 100% identical to any one of SEQ ID NOS: 2, 4, 6, 8, 16, 18, 20, and 22. In some versions, the degradation targeting sequence comprises at least one, at least two, at least three, or each of: an isoleucine at a position aligning to position 3 of any one of SEQ ID NOs: 8 and 22; a phenylalanine at a position aligning to position 20 of any one of SEQ ID NOs: 8 and 22; a cysteine at a position aligning to position 38 of any one of SEQ ID NOS: 8 and 22; and a cysteine at a position aligning to position 41 of any one of SEQ ID NOs: 8 and 22.

In some versions, the ligand moiety comprises a peptide, a nucleic acid, or a small molecule. In some versions, the ligand moiety comprises a peptide. In some versions, the ligand moiety comprises an antibody or a peptide oligomer. In some versions, the degradation targeting agent comprises a fusion protein comprising the ligand moiety fused to the degradation targeting peptide. In some versions, the ligand moiety comprises an aptamer.

In some versions, the ligand moiety binds a protein. In some versions, the ligand moiety binds an intracellular protein. In some versions, the ligand moiety binds a nuclear protein.

In some versions, the degradation targeting agent further comprises a nuclear localization signal.

Another aspect of the invention is directed to nucleic acids configured to express a degradation targeting agent of the invention.

Another aspect of the invention is directed to methods of degrading a target molecule. The methods can comprise contacting a target molecule with the degradation targeting agent of the invention.

In some versions, the contacting occurs within a cell. In some versions, the contacting occurs within a nucleus of a cell.

In some versions, the methods comprise introducing the degradation targeting agent in a cell comprising the target molecule. In some versions, the methods comprise introducing the degradation targeting agent in a nucleus of a cell comprising the target molecule. In some versions, the methods comprise introducing the degradation targeting agent within the body of a subject. In some versions, the methods comprise administering the degradation targeting agent or a nucleic acid configured to express the degradation targeting agent to a subject

The objects and advantages of the invention will appear more fully from the following detailed description of the preferred embodiment of the invention made in conjunction with the accompanying drawings.

### BRIEF DESCRIPTION OF THE DRAWINGS

FIG. 1. Recruitment of HLTF;zf-C3HC4 to Cas9 reduced Cas9 protein levels. Relative Cas9 protein levels (expressed as Cas9-ABI) in K562 cells expressing PYL1-HLTF;zf-C3HC4 or DMD negative control peptides (n=4 biological replicates; unpaired t-test; error bars represent standard deviation; ****-p<0.0001).

FIGS. 2A-2D. PYL1-HLTF;zf-C3HC4 and PYL1-RO52;zf-C3HC4 induced degradation of tagBFP-ABI in multiple cell lines within three days of recruitment. FIG. 2A. Time course of relative tagBFP-ABI levels over seven days of treatment with ABA (n=4).

FIGS. 2B-2D. Relative tagBFP-ABI levels in K562 cells (FIG. 2B), Hela cells (FIG. 2C), and HEK293T cells (FIG. 2D) after three days with (right bar at each indicated peptide) or without (left bar at each indicated peptide) treatment with ABA (n=4 biological replicates; multiple t-test; error bars represent standard deviation; *=discovery).

FIGS. 3A-3D. Effects of the E2-binding region, zinc-binding region, and C-terminus of HLTF;zf-C3HC4. FIG. 3A. Schematic of representative RING domain structure. FIG. 3B. Schematic of variants of the HLTF;zf-C3HC4 fusion peptides tested in our assay. FIG. 3C. Relative tagBFP-ABI levels in cells expressing the PYL2-HLTF;zf-C3HC4 variants or control peptides (n=4 four biological replicates; one-way ANOVA test with Dunnett's multiple comparisons test; error bars represent standard deviation; ****-p<0.0001). FIG. 3D. Relative peptide levels of the PYL2-HLTF;zf-C3HC4 variants (n=4 four biological replicates; one-way ANOVA test with Dunnett's multiple comparisons test; error bars represent standard deviation; ****-p<0.0001, ***-p<0.001, **-p<0.01).

FIGS. 4A-4D. Effect of a nuclear localization signal (NLS). FIG. 4A Relative tagBFP-ABI levels in K562 cells expressing indicated peptides with no NLS relative to control peptides (n=4 four biological replicates; one-way ANOVA test with Dunnett's multiple comparisons test; error bars represent standard deviation; ****-p<0.0001, **-p<0.01).

FIG. 4B Relative tagBFP levels in K562 cells expressing peptides with an NLS relative to control peptides (n=4 four biological replicates; one-way ANOVA test with Dunnett's multiple comparisons test; error bars represent standard deviation; ****-p<0.0001). FIG. 4C. Relative tagBFP levels in Hela cells expressing indicated peptides with no NLS relative to control peptides (n=4 four biological replicates; one-way ANOVA test with Dunnett's multiple comparisons test; error bars represent standard deviation; ****-p<0.0001, **-p<0.01). FIG. 4D. Relative BFP levels in Hela cells expressing peptides with an NLS relative to control peptides (n=4 four biological replicates; one-way ANOVA test with Dunnett's multiple comparisons test; error bars represent standard deviation; ****-p<0.0001).

FIG. 5. Decrease in target protein levels is suggested to be degradation pathway-dependent. tagBFP levels (right bar at each indicated peptide) in cells expressing indicated peptide compared to immunofluorescence staining of the Flag tag (left bar at each indicated peptide) (n=4 biological replicates; multiple t-test; error bars represent standard deviation; *=discovery).

FIGS. 6A and 6B. Tests for ubiquitin-dependence of targeted degradation by HLTF;zf-C3HC4 and RO52;zf-C3HC4. FIG. 6A. Relative tagBFP levels of peptides treated with TAK-243 (right bar at each indicated peptide) compared to untreated samples (left bar at each indicated peptide) (n=4 biological replicates; multiple t-test; error bars represent standard deviation; *=discovery). FIG. 6B. Relative tagBFP levels of peptides treated with continuous ABA (left bar at each indicated peptide), continuous ABA and TAK-243 (middle bar at each indicated peptide), and removed ABA (right bar at each indicated peptide). For each candidate peptide, each comparison between conditions had a p-value of <0.0001 (n=4 biological replicates; 2-way ANOVA; error bars represent standard deviation).

FIGS. 7A-7C. Tests for proteasome-dependence of targeted degradation by HLTF;zf-C3HC4 and RO52;zf-C3HC4. FIG. 7A. Left panel: Relative tagBFP levels of peptides incubated in ABA for three days, then treated with MG-132 for four hours compared to untreated samples. Right panel: Proportion of cells positive for BFP in a ubiquitin-tagged tagBFP (UB-R-tagBFP) construct (Dantuma N P, Lindsten K, Glas R, Jellne M, Masucci M G. Short-lived green fluorescent proteins for quantifying ubiquitin/proteasome-dependent proteolysis in living cells. Nat Biotechnol. 2000 May; 18 (5): 538-43) when treated with MG-132. FIG. 7B. Left panel: Relative tagBFP levels of peptides incubated in ABA for three days, then treated with MG-132 for 24 hours compared to untreated samples. Right panel: Proportion of cells positive for BFP in a ubiquitin-tagged tagBFP (UB-R-tagBFP) construct when treated with MG-132. FIG. 7C. Left panel: Relative tagBFP levels of peptides co-treated with ABA and MG-132 for 24 hours compared to samples treated only with ABA. Right panel: Proportion of cells positive for BFP in a ubiquitin-tagged tagBFP (UB-R-tagBFP) construct when treated with MG-132. (Left panels: n=4 biological replicates; multiple t-test; error bars represent standard deviation; *=discovery; Right panels: n=4 biological replicates; paired t-test; error bars represent standard deviation; ****-p<0.0001) In all of FIGS. 7A-7C, the left bar at each condition shows-MG-132, and the right bar at each condition shows+MG-132.

### DETAILED DESCRIPTION OF THE INVENTION

One aspects of the invention are directed to degradation targeting agents. The degradation targeting agents of the invention can comprise a degradation targeting moiety and a ligand moiety.

The degradation targeting moiety is a moiety that targets molecules bound to the agents of the invention for degradation. The degradation targeting moieties of the invention preferably comprise a degradation targeting peptide sequence. The degradation targeting peptide sequence is a sequence of the degradation targeting moiety responsible for conferring the degradation targeting activity.

In some versions, the degradation targeting peptide sequence comprises a C3HC4-type zinc-finger domain. C3HC4-type zinc-finger domains are cysteine-rich domains of 40 to 60 residues that coordinates two zinc ions. C3HC4-type zinc-finger domains generally have the consensus sequence: C-X₂-C-X₍₉₋₃₉₎-C-X₍₁₋₃₎-H-X₍₂₋₃₎-C-X₂-C-X₍₄₋₄₈₎-C-X₂-C(SEQ ID NO: 41) where X is any amino acid. C3HC4-type zinc-finger domains are well known in the art. Sec, e.g., Klug 2010 (Klug A. The discovery of zinc fingers and their development for practical applications in gene regulation and genome manipulation. Q Rev Biophys. 2010 February; 43 (1): 1-21), Hall 2005 (Hall T M. Multiple modes of RNA recognition by zinc finger proteins. Curr Opin Struct Biol. 2005 June; 15 (3): 367-73), Brown 2005 (Brown RS. Zinc finger proteins: getting a grip on RNA. Curr Opin Struct Biol. 2005 February; 15 (1): 94-8), Lorick et al. 1999 (Lorick K L, Jensen J P, Fang S, Ong A M, Hatakeyama S, Weissman A M. RING fingers mediate ubiquitin-conjugating enzyme (E2)-dependent ubiquitination. Proc Natl Acad Sci USA. 1999 Sep. 28; 96 (20): 11364-9), Gamsjaeger (Gamsjaeger R, Liew C K, Loughlin F E, Crossley M, Mackay J P. Sticky fingers: zinc-fingers as protein-recognition motifs. Trends Biochem Sci. 2007 February; 32 (2): 63-70), Laity et al. 2001 (Laity J H, Lee B M, Wright P E. Zinc finger proteins: new insights into structural and functional diversity. Curr Opin Struct Biol. 2001 February; 11 (1): 39-46), Borden et al. 1996 (Borden K L, Freemont P S. The RING finger domain: a recent example of a sequence-structure family. Curr Opin Struct Biol. 1996 June; 6 (3): 395-401), and Matthews et al. 2002 (Matthews J M, Sunde M. Zinc fingers—folds for many occasions. IUBMB Life. 2002 December; 54 (6): 351-5). The C3HC4-type zinc-finger domains of the invention in some versions comprise at least a RING domain. Exemplary C3HC4-type zinc-finger domains of the invention include SEQ ID NOS: 2, 4, 6, 8, 16, 18, 20, and 22 and peptides comprising sequences at least 80%, at least 85%, at least 90%, at least 95%, at least 96%, at least 97%, at least 98%, at least 99%, or 100% identical thereto.

In some versions, the degradation targeting peptide sequence comprises a sequence at least 80%, at least 85%, at least 90%, at least 95%, at least 96%, at least 97%, at least 98%, at least 99%, or 100% identical to any one of SEQ ID NOS: 2, 4, 6, 8, 16, 18, 20, and 22.

SEQ ID NOS: 2, 4, 6, 8 are various subsequences of the full-length HLTF (helicase-like transcription factor) protein (SEQ ID NO:23). It has been determined herein that the entire full-length HLTF protein is not required for degradation targeting. Sequences of the full-length HLTF protein not required for degradation targeting include residues 1-741 and 822-1009 of SEQ ID NO:23. In various versions of the invention, the degradation targeting moieties of the invention, or even the degradation targeting agents of the invention as a whole, are devoid of a sequence at least 80%, at least 85%, at least 90%, at least 95%, at least 96%, at least 97%, at least 98%, at least 99%, or 100% identical to residues 1-174 of SEQ ID NO:23, residues 25-174 of SEQ ID NO:23, residues 50-174 of SEQ ID NO:23, residues 75-174 of SEQ ID NO: 23, residues 100-174 of SEQ ID NO:23, residues 125-174 of SEQ ID NO:23, residues 150-174 of SEQ ID NO:23, or residues 159-174 of SEQ ID NO:23. In various versions of the invention, the degradation targeting moieties of the invention, or even the degradation targeting agents of the invention as a whole, are devoid of a sequence at least 80%, at least 85%, at least 90%, at least 95%, at least 96%, at least 97%, at least 98%, at least 99%, or 100% identical to residues 822-1009 of SEQ ID NO:23, residues 822-1000 of SEQ ID NO:23, residues 822-975 of SEQ ID NO:23, residues 822-950 of SEQ ID NO:23, residues 822-925 of SEQ ID NO:23, residues 822-900 of SEQ ID NO:23, residues 822-875 of SEQ ID NO:23, residues 822-850 of SEQ ID NO:23, or residues 822-827 of SEQ ID NO:23.

SEQ ID NOS: 16, 18, 20, and 22 are various subsequences of the full-length RO52 protein (SEQ ID NO:24). It has been determined herein that the entire full-length RO52 protein is not required for degradation targeting. Sequences of the full-length RO52 protein not required for degradation targeting include residues 1 and 82-475 of SEQ ID NO:24. In various versions of the invention, the degradation targeting moieties of the invention, or even the degradation targeting agents of the invention as a whole, are devoid of a sequence at least 80%, at least 85%, at least 90%, at least 95%, at least 96%, at least 97%, at least 98%, at least 99%, or 100% identical to residues 82-475 of SEQ ID NO:24, residues 82-450 of SEQ ID NO: 24, residues 82-425 of SEQ ID NO:24, residues 82-400 of SEQ ID NO:24, residues 82-375 of SEQ ID NO:24, residues 82-350 of SEQ ID NO:24, residues 82-325 of SEQ ID NO:24, residues 82-300 of SEQ ID NO:24, residues 82-275 of SEQ ID NO:24, residues 82-250 of SEQ ID NO:24, residues 82-225 of SEQ ID NO:24, residues 82-200 of SEQ ID NO:24, residues 82-175 of SEQ ID NO:24, residues 82-150 of SEQ ID NO:24, residues 82-125 of SEQ ID NO:24, residues 82-100 of SEQ ID NO:24, or residues 82-92 of SEQ ID NO:24.

In some versions of the invention, the degradation targeting sequence comprises at least one, at least two, at least three, or each of: a residue other than alanine at a position aligning to position 3 of any one of SEQ ID NOs: 8 and 22 or a corresponding position in any one of SEQ ID NOS: 2, 4, 6, 16, 18, and 20; a residue other than alanine at a position aligning to position 20 of any one of SEQ ID NOs: 8 and 22 or a corresponding position in any one of SEQ ID NOS: 2, 4, 6, 16, 18, and 20; a residue other than alanine at a position aligning to position 38 of any one of SEQ ID NOS: 8 and 22 or a corresponding position in any one of SEQ ID NOS: 2, 4, 6, 16, 18, and 20; and a residue other than alanine at a position aligning to position 41 of any one of SEQ ID NOs: 8 and 22 or a corresponding position in any one of SEQ ID NOS: 2, 4, 6, 16, 18, and 20. In some versions of the invention, the degradation targeting sequence comprises at least one, at least two, at least three, or each of: an isoleucine or a conservative variant thereof at a position aligning to position 3 of any one of SEQ ID NOs: 8 and 22 or a corresponding position in any one of SEQ ID NOS: 2, 4, 6, 16, 18, and 20; a phenylalanine or a conservative variant thereof at a position aligning to position 20 of any one of SEQ ID NOs: 8 and 22 or a corresponding position in any one of SEQ ID NOS: 2, 4, 6, 16, 18, and 20; a cysteine or a conservative variant thereof at a position aligning to position 38 of any one of SEQ ID NOS: 8 and 22 or a corresponding position in any one of SEQ ID NOS: 2, 4, 6, 16, 18, and 20; and a cysteine or a conservative variant thereof at a position aligning to position 41 of any one of SEQ ID NOs: 8 and 22 or a corresponding position in any one of SEQ ID NOS: 2, 4, 6, 16, 18, and 20. Corresponding positions in SEQ ID NOS: 2, 4, 6, 16, 18, and 20 can be found by aligning SEQ ID NOS: 8 or 22 with SEQ ID NOS: 2, 4, 6, 16, 18, and 20. In some versions of the invention, the degradation targeting sequence comprises at least one, at least two, at least three, or each of: an isoleucine at a position aligning to position 3 of any one of SEQ ID NOs: 8 and 22 or a corresponding position in any one of SEQ ID NOS: 2, 4, 6, 16, 18, and 20; a phenylalanine at a position aligning to position 20 of any one of SEQ ID NOs: 8 and 22 or a corresponding position in any one of SEQ ID NOS: 2, 4, 6, 16, 18, and 20; a cysteine at a position aligning to position 38 of any one of SEQ ID NOS: 8 and 22 or a corresponding position in any one of SEQ ID NOS: 2, 4, 6, 16, 18, and 20; and a cysteine at a position aligning to position 41 of any one of SEQ ID NOs: 8 and 22 or a corresponding position in any one of SEQ ID NOS: 2, 4, 6, 16, 18, and 20. Corresponding positions in SEQ ID NOS: 2, 4, 6, 16, 18, and 20 can be found by aligning SEQ ID NOS: 8 or 22 with SEQ ID NOS: 2, 4, 6, 16, 18, and 20.

The ligand moiety can comprise any moiety that binds to a target molecule (e.g., a molecule targeted for degradation). The ligand moiety can take any molecular form. Examples include peptides, nucleic acids, small molecules, nanoparticles, microparticles, or any combination thereof.

“Peptide,” “polypeptide,” and “protein” are used interchangeably herein to refer to a chain of amino acids linked by peptide bonds.

“Nucleic acid” is used herein to refer to a chain of linked nucleotides. Examples include deoxynucleic acids (DNAs) and ribonucleic acids (RNAs), which comprise nucleotides linked with a phosphodiester linkage, and xeno nucleic acids (XNAs), which comprise nucleotides linked with linkages other than phosphodiester linkages.

“Small molecule” is used herein to refer to an organic compound having a molecular weight of 5,000 daltons or less, such as 4,500 daltons or less, 4,000 daltons or less, 3,500 daltons or less, 3,000 daltons or less, 2,500 daltons or less, 2,000 daltons or less, 1,500 daltons or less, or 1,000 daltons or less.

In some versions, the ligand moiety comprises a peptide oligomer. As used herein, peptide oligomer refers to a peptide having 50 or fewer amino acids linked by peptide bonds, such as 45 or fewer, 40 or fewer, 35 or fewer, 30 or fewer, 25 or fewer, 20 or fewer, 15 or fewer, 10 or fewer, or 5 or fewer amino acids linked by peptide bonds. Peptide oligomers suitable for use in targeted protein degradation are well known in the art. See, e.g., Jiang et al. 2019 (Jiang Z, Guan J, Qian J, Zhan C. Peptide ligand-mediated targeted drug delivery of nanomedicines. Biomater Sci. 2019 Jan. 29; 7 (2): 461-471), Zhao et al. 2022 (Zhao L, Zhao J, Zhong K, Tong A, Jia D. Targeted protein degradation: mechanisms, strategies and application. Signal Transduct Target Ther. 2022 Apr. 4; 7 (1): 113), and Békés et al. 2022 (Békés M, Langley DR, Crews CM. PROTAC targeted protein degraders: the past is prologue. Nat Rev Drug Discov. 2022 March; 21 (3): 181-200), among others. Peptide oligomers suitable for binding a specific target can be designed using methods known in the art. See, e.g., Gruber et al. 2010 (Gruber CW, Muttenthaler M, Freissmuth M. Ligand-based peptide design and combinatorial peptide libraries to target G protein-coupled receptors. Curr Pharm Des. 2010; 16 (28): 3071-88).

In some versions, the ligand moiety comprises an antibody. The term “antibody” includes antibodies or immunoglobulins of any isotype (e.g., IgG (e.g., IgG1, IgG2, IgG3 or IgG4), IgE, IgD, IgA, IgM, etc.); whole antibodies (e.g., antibodies composed of a tetramer which in turn is composed of two dimers of a heavy and light chain polypeptide); single chain antibodies; fragments of antibodies (e.g., fragments of whole or single chain antibodies) which retain specific binding to the membrane or extracellular protein, including, but not limited to, Fv, single chain Fv (scFv), Fab, F(ab′)2, Fab′, (scFv′) 2, diabodies, and nanobodies; chimeric antibodies; monoclonal antibodies; fully human antibodies; humanized antibodies (e.g., humanized whole antibodies, humanized antibody fragments, etc.); camelid antibodies; intrabodies; and fusion proteins including an antigen-binding portion of an antibody and a non-antibody protein or fragment thereof. The generation and use of antibodies in binding molecular targets is well known in the art. Methods for generating antibodies against any target molecule are also well known in the art. Sec, e.g., U.S. Pat. No. 11,673,954 B2, U.S. Pat. No. 11,560,427 B2, U.S. Pat. No. 11,298,433 B2, US 2010/0143371 A1, among others.

In some versions, the ligand moiety comprises an aptamer. As used herein, “aptamer” refers to a nucleic acid (e.g., ssDNA, RNA, XNA) that binds a specific target molecule or family of target molecules. In some versions, the aptamer is a nucleic acid oligomer. Aptamers for any target molecule can be made by systematic evolution of ligands by exponential enrichment (SELEX). Sec, e.g., Gold 2015 (Gold L. SELEX: How It Happened and Where It will Go. J Mol Evol. 2015 December; 81 (5-6): 140-3) and references cited therein.

The linkage between the ligand moiety and the degradation targeting moiety can be via any peptide or chemical linkage.

In cases where the ligand moiety comprises a peptide, the linkage can be via any peptide-peptide linkage. A number of peptide-peptide linkages are known in the art. In some versions, the linkage occurs via the expression of the degradation targeting agent as a fusion protein comprising the ligand moiety and a degradation targeting moiety. The ligand moiety and a degradation targeting moiety can be linked in the fusion protein either directly or via a peptide linker. The degradation targeting moiety can be linked (either directly or via a peptide linker) to the ligand moiety at the N-terminus of the degradation targeting moiety or the C-terminus of the degradation targeting moiety. Similarly, the ligand moiety can be linked (either directly or via a peptide linker) to the degradation targeting moiety at the N-terminus of the ligand moiety or the C-terminus of the ligand moiety.

The peptide linker can be of any suitable length. In some embodiments, the peptide linker is at least about any of 1, 2, 3, 4, 5, 6, 7, 8, 9, 10, 11, 12, 13, 14, 15, 16, 17, 18, 19, 20, 25, 30, 35, 40, 50, 75, 100, or more amino acids long. In some embodiments, the peptide linker is no more than about 100, 75, 50, 40, 35, 30, 25, 20, 19, 18, 17, 16, 15, 14, 13, 12, 11, 10, 9, 8, 7, 6, 5, or fewer amino acids long. In some embodiments, the length of the peptide linker is any of about 1 amino acid to about 10 amino acids, about 1 amino acid to about 20 amino acids, about 1 amino acid to about 30 amino acids, about 5 amino acids to about 15 amino acids, about 10 amino acids to about 25 amino acids, about 5 amino acids to about 30 amino acids, about 10 amino acids to about 30 amino acids long, about 30 amino acids to about 50 amino acids, about 50 amino acids to about 100 amino acids, or about 1 amino acid to about 100 amino acids.

The peptide linker may have a naturally occurring sequence, or a non-naturally occurring sequence. In some embodiments, the peptide linker is a flexible linker. Exemplary flexible linkers include glycine polymers (G)ₙ, glycine-serine polymers (including, for example, (GS)ₙ, (GSGGS (SEQ ID NO:29))ₙ, (GGGS (SEQ ID NO:30))ₙ, and (GGGGS (SEQ ID NO: 31))ₙ, where n is an integer of at least one, such as 1, 2, 3, 4, 5, 6, 7, 8, 9, 10, 15, 20, 25 or more, and/or, optionally, up to 10, 15, 20, 25, 30, 35, or more), glycine-alanine polymers, alanine-serine polymers, and other flexible linkers known in the art. Exemplary peptide linkers can include peptide sequences such as GGSGG (SEQ ID NO:32), GGGGSGGGS (SEQ ID NO: 33), GGGGSGGGGSGGGGS (SEQ ID NO:34), GPGGP (SEQ ID NO:35), AALVGPGGQGGGGSGGGGSGGGGSGGGGSGGGGSMA (SEQ ID NO:36), EPKSSDKTHTSPPSP (SEQ ID NO:37), and GPGGQGTGPGGS (SEQ ID NO:38). Other suitable peptide linkers are provided in Klein et al. 2014 (Klein JS, Jiang S, Galimidi RP, Keeffe JR, Bjorkman PJ. Design and characterization of structured protein linkers with differing flexibilities. Protein Eng Des Sel. 2014 October; 27 (10): 325-30) and Reddy Chichili et al. 2013 (Reddy Chichili VP, Kumar V, Sivaraman J. Linkers in the structural biology of protein-protein interactions. Protein Sci. 2013 February; 22 (2): 153-67).

In some versions, the ligand moiety and the degradation targeting moiety are linked via a chemical crosslinker. A large number of chemical crosslinkers suitable for crosslinking proteins to proteins or proteins to small molecules or aptamers are known in the art.

Various exemplary linkers include ester linkers (e.g., N-hydroxysuccinimide (NHS) ester, sulfo-NHS ester or PFP ester or thioester), amide linkers, maleimide or maleimide-based linkers, valinc-citrulline linkers, hydrazone linkers, N-succinimidyl-4-(2-pyridyldithio) butyrate (SPDB) linkers, succinimidyl-4-(A/-maleimidomethyl)cyclohexane-1-carboxylate (SMCC) linkers, vinylsulfone-based linkers, oxime-based linkers, carbonate-based linkers, ester-based linkers, linkers that include polyethylene glycol (PEG), such as, but not limited to tetracthylene glycol, linkers that include propanoic acid, linkers that include caproleic acid, and linkers including any combination thereof. In one embodiment, the linker is PEG. Various lengths of PEG may be used as linkers, such as PEG3, PEG 12, etc. Various types of linkers are described in Ducry et al. 2010 (Ducry L, Stump B. Antibody-drug conjugates: linking cytotoxic payloads to monoclonal antibodies. Bioconjug Chem. 2010 January; 21 (1): 5-13) and Liu et al. 2022 (Liu Z, Hu M, Yang Y, Du C, Zhou H, Liu C, Chen Y, Fan L, Ma H, Gong Y, Xie Y. An overview of PROTACs: a promising drug discovery paradigm. Mol Biomed. 2022 Dec. 20; 3 (1): 46).

The target molecules to which the ligand moiety binds can include any type of target molecule. Exemplary target molecules include proteins, lipids, nucleic acids, or other types of molecules, including non-proteinaceous cell-membrane components. For the purposes of this disclosure, cellular organelles such as a mitochondrion, an endoplasmic reticulum, a Golgi apparatus, etc., and/or components (e.g., proteins) associated therewith constitute a “target molecule” for targeting for degradation. Sec, e.g., Takahashi et al. 2019 (Takahashi D, Moriyama J, Nakamura T, Miki E, Takahashi E, Sato A, Akaike T, Itto-Nakama K, Arimoto H. AUTACs: Cargo-Specific Degraders Using Selective Autophagy. Mol Cell. 2019 Dec. 5; 76 (5): 797-810.e10). In exemplary versions, the target molecule comprises a protein (a “target protein”). In some versions, the target protein comprises an intracellular protein. “Intracellular protein” refers to a protein that has at least a portion that is at least temporarily present within a cell. In some versions, the target protein comprises a nuclear protein. “Intracellular protein” refers to a protein that has at least a portion that is at least temporarily present within a nucleus of a cell. In some versions, the target protein comprises a membrane protein. In some versions, the target protein comprises a transmembrane protein. In some versions, the target protein comprises a peripheral membrane protein. In some versions, the target protein comprises an extracellular protein.

Non-limiting, exemplary target proteins include AKT, alpha-synuclein, SNCA, NACP, alpha-tubulin (TUBA), AXL, UFO, BCL2, BCL-xL, beta-tubulin (TUBB), BLK, BRD2, BD2, BTK, Cdc20, p55CDC, CDK2, CDKN2, CDK4, PSK-J3, CRBN, CYP1B1, EED, EGFR, ERBB, HER1, EZH2, KMT6, ENX-1, FAK, PTK2, FAKI, HDAC3, IDO1, MDM2, Tau, VHL, pVHL, Wec1, WEE1hu, and HER2.

In certain embodiments, the target protein comprises a membrane receptor. Membrane receptors of interest include, but are not limited to, stem cell receptors, immune cell receptors, growth factor receptors, cytokine receptors, hormone receptors, receptor tyrosine kinases, a receptor in the epidermal growth factor receptor (EGFR) family (e.g., HER2 (human epidermal growth factor receptor 2), etc.), a receptor in the fibroblast growth factor receptor (FGFR) family, a receptor in the vascular endothelial growth factor receptor (VEGFR) family, a receptor in the platelet derived growth factor receptor (PDGFR) family, a receptor in the rearranged during transfection (RET) receptor family, a receptor in the Eph receptor family, a receptor in the discoidin domain receptor (DDR) family, and a mucin protein (e.g., MUCI). In some versions, the target protein is EGFR, which is known to be frequently mutated or overexpressed in different types of human cancers (Yarden and Pines, 2012, Nat Rev Cancer. 12:553-563; Sigismund et al., 2018, Mol. Oncol. 12:3-20).

In some versions, the target protein comprises an immune inhibitory receptor. As used herein, an “immune inhibitory receptor” is a receptor present on an immune cell that negatively regulates an immune response. Examples of inhibitory immune receptors include immune inhibitory receptors of the Ig superfamily, including but not limited to: CD200R, CD300a (IRp60; mouse MAIR-1), CD300f (IREM-1), CEACAMI (CD66a), FcγRIIb, ILT-2 (LIR-1; LILRB1; CD85j), ILT-3 (LIR-5; CD85k; LILRB4), ILT-4 (LIR-2; LILRB2), ILT-5 (LIR-3; LILRB3; mouse PIR-B); LAIR-1, PECAM-1 (CD31), PILR-α (FDF03), SIRL-1, and SIRP-α. Further examples of immune inhibitory receptors include sialic acid-binding Ig-like lectin (Siglec) receptors, e.g., Siglec 7, Siglec9, and/or the like. Additional examples of immune inhibitory receptors include C-type lectins, including but not limited to: CLEC4A (DCIR), Ly49Q and MICL. Details regarding immune inhibitory receptors may be found, e.g., in Steevels et al., 2011, Eur. J. Immunol. 4:575-587.

In some versions, the target protein comprises a ligand of an immune inhibitory receptor, one example of which is CD47, which binds to SIRP-α to prevent phagocytosis and known to be overexpressed in cancer cells (Eladl et al., 2020, J. Hematol. Oncol. 13:96).

In some versions, the target protein comprises an immune checkpoint molecule, including immune checkpoint proteins and ligands. Non-limiting examples of immune checkpoint molecules include PD-1, PD-L1, CTLA4, TIM3, LAG3, TIGIT, and a member of the B7 family. In one embodiment, the membrane protein is PD-L1 (Programmed Cell Death Ligand 1), which binds PD-1 (programmed cell death-1) to inhibit apoptosis and known to be overexpressed in cancer cells (Yi et al., 2021, J. Hematol. Oncol. 14:10).

In some versions, the target protein comprises a ligand for a membrane receptor. Membrane receptor ligands of interest include, but are not limited to, growth factors (e.g., epidermal growth factor (EGF), vascular endothelial growth factor (VEGF), and the like), cytokines (e.g., an interleukin, an interferon, a tumor necrosis factor (TNF), a transforming growth factor β (TGF-β), including any particular subtypes of such cytokines), hormones, and the like.

In some versions, the target protein comprises an antibody e.g., an antibody that binds a membrane protein or a different extracellular protein. The antibody may be an auto-antibody. By “auto-antibody” is meant an antibody produced by the immune system that is directed against one or more of the individual's own proteins. Cancer cells can induce an immunological response resulting in the production of tumor-associated auto-antibodies. Non-limiting examples of auto-antibodies include rheumatoid factor (RF), antinuclear antibody (ANA), antineutrophil cytoplasmic antibodies (ANCA), anti-double stranded DNA (anti-dsDNA), anticentromere antibodies (ACA), anticyclic citrullinated peptide antibodies (anti-CCP), extractable nuclear antigen antibodies (ENA), anticardiolipin antibodies, beta-2 glycoprotein 1 antibodies, antiphospholipid antibodies (APA), lupus anticoagulants (LA), anti-tissue transglutaminase (anti-tTG), anti-gliadin antibodies (AGA), intrinsic factor antibodies, parietal cell antibodies, thyroid antibodies, smooth muscle antibodies (SMA), antimitochondrial antibodies (AMA), anti-glomerular basement membrane (GBM), acetylcholine receptor (AChR) antibodies, etc.

In some versions, the target protein comprises a secreted protein, including, but not limited to, secreted growth factors, extracellular matrix-degrading proteinases, cell motility factors and immunoregulatory cytokines or other bioactive molecules.

In some versions, the target protein comprises a constituent protein of a cellular organelle, such as a mitochondrion, an endoplasmic reticulum, a Golgi apparatus, etc. See, e.g., Takahashi et al. 2019 (Takahashi D, Moriyama J, Nakamura T, Miki E, Takahashi E, Sato A, Akaike T, Itto-Nakama K, Arimoto H. AUTACs: Cargo-Specific Degraders Using Selective Autophagy. Mol Cell. 2019 Dec. 5; 76 (5): 797-810.e10).

In some versions, the degradation targeting agents of the invention can further comprise a nuclear localization signal (NLS), sometimes also referred to in the art as a nuclear localization sequence. An NLS is an amino acid sequence that “tags” a protein for import into the cell nucleus by nuclear transport. NLSs are generally short peptides that act as a signal fragment that mediates the transport of proteins from the cytoplasm into the nucleus. NLSs can be classified as classical NLSs and non-classical NLSs, among others. Classical NLSs encompass two categories, termed “monopartite” and “bipartite.”. Monopartite NLSs comprise a single cluster composed of 4-8 basic amino acids, which generally contains 4 or more positively charged residues, that is, arginine (R) or lysine (K). The characteristic motif of monopartite NLSs are usually defined as K(K/R)X(K/R), where X can be any residue. For example, the NLS of SV40 large T-antigen is PKKKRKV (SEQ ID NO:39), with five consecutive positively charged amino acids (KKKRK (SEQ ID NO:40)). Non-classical NLSs are neither similar to canonical signals nor rich in arginine or lysine residues. Among non-classical NLSs are the “proline-tyrosine” types, named PY-NLS. PY-NLS is characterized by 20-30 amino acids that assume a disordered structure, consisting of N-terminal hydrophobic or basic motifs and C-terminal R/K/H(X)₂₋₅PY motifs (where X₂₋₅ is any sequence of 2-5 residues). Other types of NLSs exist. NLSs are described in detail in Lu et al. 2021 (Lu J, Wu T, Zhang B, Liu S, Song W, Qiao J, Ruan H. Types of nuclear localization signals and mechanisms of protein import into the nucleus. Cell Commun Signal. 2021 May 22; 19 (1): 60). Exemplary NLSs or types of NLSs of the invention include any described in Lu et al. 2021 or otherwise known in the art. The NLS can be linked to the ligand moiety and the degradation targeting moiety using any suitable linker, including any described herein, in any location on the degradation targeting agent. If the degradation targeting agent is a fusion protein, the NLS can be N-terminal to both the ligand moiety and the degradation targeting moiety, C-terminal to both the ligand moiety and the degradation targeting moiety, or between the ligand moiety and the degradation targeting moiety.

In some versions, the cell comprising the target molecule is a cancer cell. By “cancer cell” is meant a cell exhibiting a neoplastic cellular phenotype, which may be characterized by one or more of, for example, abnormal cell growth, abnormal cellular proliferation, loss of density-dependent growth inhibition, anchorage-independent growth potential, ability to promote tumor growth and/or development in an immunocompromised non-human animal model, and/or any appropriate indicator of cellular transformation. “Cancer cell” may be used interchangeably herein with “tumor cell”, “malignant cell,” “neoplastic cell,” or “cancerous cell”, and encompasses cancer cells of a solid tumor, a semi-solid tumor, a hematological malignancy (e.g., a leukemia cell, a lymphoma cell, a myeloma cell, etc.), a primary tumor, a metastatic tumor, and the like. In some embodiments, the target protein comprised by the cancer cell is a tumor-associated antigen or a tumor-specific antigen. Exemplary cancer cells include lung cancer cells (e.g., non-small cell lung cancer (NSCLC)), gastric cancer cells, colon cancer cells, heart cancer cells, neck cancer cells, breast cancer cells, melanoma cells (e.g., metastatic malignant melanoma), renal cancer cells (e.g. clear cell carcinoma), prostate cancer cells (e.g. hormone refractory prostate adenocarcinoma), bone cancer cells, pancreatic cancer cells, skin cancer cells, cutaneous or intraocular malignant melanoma cells, uterine cancer cells, ovarian cancer cells, rectal cancer cells, cancer cells of the anal region, stomach cancer cells, testicular cancer cells, uterine cancer cells, cancer cells of the fallopian tubes, cancer cells of the endometrium, cancer cells of the cervix, cancer cells of the vagina, cancer cells of the vulva, Hodgkin's Disease cells, non-Hodgkin's lymphoma cells, cancer cells of the esophagus, cancer cells of the small intestine, cancer cells of the endocrine system, cancer cells of the thyroid gland, cancer cells of the parathyroid gland, cancer cells of the adrenal gland, cancer cells of the urethra, cancer cells of the penis, chronic or acute leukemia cells including acute myeloid leukemia cells, chronic myeloid leukemia cells, acute lymphoblastic leukemia cells, chronic lymphocytic leukemia cells, lymphocytic lymphoma cells, cancer cells of the bladder, cancer cells of the kidney or ureter, cancer cells of the renal pelvis, neoplastic cells of the central nervous system (CNS), primary CNS lymphoma cells, spinal axis tumor cells, brain stem glioma cells, pituitary adenoma cells, Kaposi's sarcoma cells, epidermoid cancer cells, squamous cell cancer cells, gastrointestinal carcinoid tumor cells, colorectal cancer cells, gastrointestinal stromal tumor cells, Leiomyosarcoma cells, and T-cell lymphoma cells.

Another aspect of the invention is directed to nucleic acids configured to express the degradation targeting agents of the invention, such as degradation targeting agents in the form of fusion proteins. The nucleic acid configured to express the degradation targeting agents of the invention can comprise any type of nucleic acid, such as DNA or RNA (e.g., mRNA), capable of being translated or transcribed and translated into one or more peptides. The delivery of nucleic acids intracellularly or in vivo for therapeutic applications is well known in the art.

In some versions, a nucleic acid configured to express the degradation targeting agent can be cloned into an expression vector. Methods commonly known in the art of recombinant DNA technology which can be used are described in Ausubel et al. (eds.), 1993, Current Protocols in Molecular Biology, John. Wiley & Sons, NY; and Kriegler, 1990, Gene Transfer and Expression, A Laboratory Manual, Stockton Press, N.Y.

A DNA encoding the degradation targeting agent may be recombinantly engineered into a variety of host vector systems that also provide for replication of the DNA in large scale and contain the necessary elements for directing the transcription. The use of such a vector to transfect target cells will result in transcription of sufficient amounts of the degradation targeting agent to affect a cellular process. For example, a vector can be introduced in vivo such that it is taken up by a cell and directs the transcription of the degradation targeting agent. Such a vector can remain episomal or become chromosomally integrated, as long as it can be expressed to produce the desired degradation targeting agent. Such vectors can be constructed by recombinant DNA technology methods standard in the art.

Vectors encoding the degradation targeting agent can be plasmid, viral, or others known in the art, used for replication and expression in mammalian cells. Expression of the sequence encoding the degradation targeting agent can be regulated by any promoter/enhancer sequences known in the art to act in mammalian, preferably human cells. Such promoters/enhancers can be inducible or constitutive. Such promoters include but are not limited to the SV40 early promoter region (Benoist, C. and Chambon, P. 1981, Nature 290:304-310), the promoter contained in the 3′ long terminal repeat of Rous sarcoma virus (Yamamoto et al., 1980, Cell 22:787-797), the herpes thymidine kinase promoter (Wagner ct al., 1981, Proc. Natl. Acad. Sci. U.S.A. 78:1441-1445), the regulatory sequences of the metallothionein gene (Brinster et al., 1982, Nature 296:39-42), the viral CMV promoter, the human β-chorionic gonadotropin-6 promoter (Hollenberg et al., 1994, Mol. Cell. Endocrinology 106:111-119), etc. In one embodiment, cell type specific promoter/enhancer sequences may be used to promote the synthesis of the degradation targeting agent in particular cells or tissue types.

Vectors for use in the practice of the invention include any eukaryotic expression vectors, including but not limited to viral expression vectors such as those derived from the class of retroviruses, lentiviruses, adenoviruses, or adeno-associated viruses.

Nucleic acids comprising a sequence encoding a degradation targeting agent of the invention can be administered by way of nucleic acid (e.g., gene, plasmid, mRNA, etc.) delivery and expression into a host cell. Any of the methods for nucleic acid delivery into a host cell available in the art can be used according to the present invention. For general reviews of the methods of gene delivery see Strauss, M. and Barranger, J. A., 1997, Concepts in Gene Therapy, by Walter de Gruyter & Co., Berlin; Goldspiel et al., 1993, Clinical Pharmacy 12:488-505; Wu and Wu, 1991, Biotherapy 3:87-95; Tolstoshev, 1993, Ann. Rev. Pharmacol. Toxicol. 33:573-596; Mulligan, 1993, Science 260:926-932; and Morgan and Anderson, 1993, Ann. Rev. Biochem. 62:191-217; 1993, TIBTECH 11 (5): 155-215. Examples of nucleic acids include DNA, mRNA, etc.

In some embodiments, the nucleic acid encoding a degradation targeting agent of the invention is directly administered in vivo, under conditions effective for production of the protein. This can be accomplished by any of numerous methods known in the art, e.g., by constructing it as part of an appropriate nucleic acid expression vector and administering it so that it becomes intracellular, e.g., by infection using a defective or attenuated retroviral or other viral vector (see U.S. Pat. No. 4,980,286), or by direct injection of naked DNA, or by use of microparticle bombardment (e.g., a gene gun; Biolistic, Dupont), or coating with lipids or cell-surface receptors or transfecting agents, encapsulation in liposomes, microparticles, or microcapsules, or by administering it in linkage to a peptide which is known to enter the nucleus, or by administering it in linkage to a ligand subject to receptor-mediated endocytosis (scc e.g., Wu and Wu, 1987, J. Biol. Chem. 262:4429-4432).

In a specific embodiment, a viral vector that contains sequences encoding a degradation targeting agent of the invention can be used. For example, a retroviral vector can be utilized that has been modified to delete retroviral sequences that are not necessary for packaging of the viral genome and integration into host cell DNA (see Miller et al., 1993, Meth. Enzymol. 217:581-599). Alternatively, adenoviral or adeno-associated viral vectors can be used for gene delivery to cells or tissues. (See, Kozarsky and Wilson, 1993, Current Opinion in Genetics and Development 3:499-503 for a review of adenovirus-based gene delivery).

In some embodiments of the invention, an adeno-associated viral vector may be used to deliver nucleic acid molecules that encode a degradation targeting agent of the invention. The vector is designed so that, depending on the level of expression desired, a promoter and/or enhancer element of choice may be inserted into the vector.

Another approach to nucleic acid delivery involves transferring the nucleic acid to cells in tissue culture by such methods as electroporation, lipofection, calcium phosphate mediated transfection, or viral infection. Usually, the method of transfer includes the transfer of a selectable marker to the cells. The cells are then placed under selection to isolate those cells that have taken up and are expressing the transferred gene. The resulting recombinant cells can be delivered to a host by various methods known in the art. In a preferred embodiment, the cell used for gene delivery is autologous to the host cell.

In some versions, the degradation targeting agents of the invention administered as pre-expressed proteins to a cell or subject. Methods for delivering proteins intracellular to cells are described by Yu et al. 2021 (Yu S, Yang H, Li T, Pan H, Ren S, Luo G, Jiang J, Yu L, Chen B, Zhang Y, Wang S, Tian R, Zhang T, Zhang S, Chen Y, Yuan Q, Ge S, Zhang J, Xia N. Efficient intracellular delivery of proteins by a multifunctional chimaeric peptide in vitro and in vivo. Nat Commun. 2021 Aug. 26; 12 (1): 5131), among others.

The degradation targeting agents of the invention and/or the nucleic acid configured to express same (collectively, “pharmaceutical agents”) can be administered in the form of a composition, such as a pharmaceutical composition. The pharmaceutical compositions can optionally include a pharmaceutically acceptable carrier, excipient, and/or stabilizer. The pharmaceutical compositions can be prepared by mixing a pharmaceutical agent having the desired degree of purity with optional pharmaceutically acceptable carriers, excipients or stabilizers (Remington's Pharmaceutical Sciences 16th edition, Osol, A. Ed. (1980)), in the form of lyophilized formulations or aqueous solutions.

Acceptable carriers, excipients, or stabilizers are nontoxic to recipients at the dosages and concentrations employed, and include buffers, antioxidants including ascorbic acid, methionine, Vitamin E, sodium metabisulfite; preservatives, isotonicifiers (e.g. sodium chloride), stabilizers, metal complexes (e.g. Zn-protein complexes); chelating agents such as EDTA and/or non-ionic surfactants.

Examples of physiologically acceptable carriers include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptide; proteins, such as serum albumin, gelatin, or immunoglobulins; hydrophilic polymers such as polyvinylpyrrolidone; amino acids such as glycine, glutamine, asparagine, arginine or lysine;

monosaccharides, disaccharides, and other carbohydrates including glucose, mannose, or dextrins; chelating agents such as EDTA; sugars such as sucrose, mannitol, trehalose or sorbitol; salt-forming counterions such as sodium; metal complexes (e.g. Zn-protein complexes); and/or nonionic surfactants such as TWEEN™, polyethylene glycol (PEG), and PLURONICS™ or polyethylene glycol (PEG).

Buffers are used to control the pH in a range which optimizes the therapeutic effectiveness, especially if stability is pH dependent. Buffers are preferably present at concentrations ranging from about 50 mM to about 250 mM. Suitable buffering agents for use in the present application include both organic and inorganic acids and salts thereof. For example, citrate, phosphate, succinate, tartrate, fumarate, gluconate, oxalate, lactate, acetate. Additionally, buffers may comprise histidine and trimethylamine salts such as Tris.

Preservatives are added to retard microbial growth and are typically present in a range from 0.2%-1.0% (w/v). The addition of a preservative may, for example, facilitate the production of a multi-use (multiple-dose) formulation. Suitable preservatives for use in the present application include octadecyldimethylbenzyl ammonium chloride; hexamethonium chloride; benzalkonium halides (e.g., chloride, bromide, iodide), benzethonium chloride; thimerosal, phenol, butyl or benzyl alcohol; alkyl parabens such as methyl or propyl paraben; catechol; resorcinol; cyclohexanol, 3-pentanol, and m-cresol.

Tonicity agents, sometimes known as “stabilizers” are present to adjust or maintain the tonicity of liquid in a composition. When used with large, charged biomolecules such as proteins and antibodies, they are often termed “stabilizers” because they can interact with the charged groups of the amino acid side chains, thereby lessening the potential for inter and intra-molecular interactions. Tonicity agents can be present in any amount between 0.1% to 25% by weight, preferably 1% to 5%, taking into account the relative amounts of the other ingredients. Preferred tonicity agents include polyhydric sugar alcohols, preferably trihydric or higher sugar alcohols, such as glycerin, erythritol, arabitol, xylitol, sorbitol and mannitol.

Additional excipients include agents which can serve as one or more of the following: (1) bulking agents, (2) solubility enhancers, (3) stabilizers and (4) and agents preventing denaturation or adherence to the container wall. Such excipients include: polyhydric sugar alcohols (enumerated above); amino acids such as alanine, glycine, glutamine, asparagine, histidine, arginine, lysine, ornithine, leucine, 2-phenylalanine, glutamic acid, threonine, etc.; organic sugars or sugar alcohols such as sucrose, lactose, lactitol, trehalose, stachyose, mannose, sorbose, xylose, ribose, ribitol, myoinisitose, myoinisitol, galactose, galactitol, glycerol, cyclitols (e.g., inositol), polyethylene glycol; sulfur containing reducing agents, such as urea, glutathione, thioctic acid, sodium thioglycolate, thioglycerol, α-monothioglycerol and sodium thio sulfate; low molecular weight proteins such as human serum albumin, bovine serum albumin, gelatin or other immunoglobulins; hydrophilic polymers such as polyvinylpyrrolidone; monosaccharides (e.g., xylose, mannose, fructose, glucose; disaccharides (e.g., lactose, maltose, sucrose); trisaccharides such as raffinose; and polysaccharides such as dextrin or dextran.

Non-ionic surfactants or detergents (also known as “wetting agents”) are present to help solubilize the therapeutic agent as well as to protect the therapeutic protein against agitation-induced aggregation, which also permits the formulation to be exposed to shear surface stress without causing denaturation of the active therapeutic protein or antibody. Non-ionic surfactants are present in a range of about 0.05 mg/ml to about 1.0 mg/ml, preferably about 0.07 mg/ml to about 0.2 mg/ml.

Suitable non-ionic surfactants include polysorbates (20, 40, 60, 65, 80, etc.), polyoxamers (184, 188, etc.), PLURONIC® polyols, TRITON®, polyoxyethylene sorbitan monoethers (TWEEN®-20, TWEEN®-80, etc.), lauromacrogol 400, polyoxyl 40 stearate, polyoxyethylene hydrogenated castor oil 10, 50 and 60, glycerol monostearate, sucrose fatty acid ester, methyl celluose and carboxymethyl cellulose. Anionic detergents that can be used include sodium lauryl sulfate, dioctyle sodium sulfosuccinate and dioctyl sodium sulfonate. Cationic detergents include benzalkonium chloride or benzethonium chloride.

In order for the pharmaceutical compositions to be used for in vivo administration, they should be sterile. The pharmaceutical composition may be rendered sterile by filtration through sterile filtration membranes. The pharmaceutical compositions herein generally are placed into a container having a sterile access port, for example, an intravenous solution bag or vial having a stopper pierceable by a hypodermic injection needle.

The route of administration is in accordance with known and accepted methods, such as by single or multiple bolus or infusion over a long period of time in a suitable manner, e.g., injection or infusion by subcutaneous, intravenous, intraperitoneal, intramuscular, intra-arterial, intralesional or intraarticular routes, topical administration, inhalation or by sustained release or extended-release means. In some embodiments, the pharmaceutical composition is administered locally.

The pharmaceutical agents can be administered in accord with known methods, such as intravenous administration as a bolus or by continuous infusion over a period of time, by intramuscular, intraperitoneal, intracerobrospinal, subcutaneous, intravenous (i.v.), intraarticular, intrasynovial, intrathecal, oral, topical, or inhalation routes. A reconstituted formulation can be prepared by dissolving a lyophilized pharmaceutical agent described herein in a diluent such that the agent is dispersed throughout. Exemplary pharmaceutically acceptable (safe and non-toxic for administration to a human) diluents suitable for use in the present application include, but are not limited to, sterile water, bacteriostatic water for injection (BWFI), a pH buffered solution (e.g. phosphate-buffered saline), sterile saline solution, Ringer's solution or dextrose solution, or aqueous solutions of salts and/or buffers.

In some embodiments, the pharmaceutical agents can be administered to the individual by subcutaneous (i.e. beneath the skin) administration. For such purposes, the pharmaceutical agents may be injected using a syringe. However, other devices for administration of the pharmaceutical agents are available such as injection devices; injector pens; auto-injector devices, needleless devices; and subcutaneous patch delivery systems.

In some embodiments, the pharmaceutical agents can be administered to the individual intravenously. In some embodiments, the pharmaceutical agent is administered to an individual by infusion, such as intravenous infusion. Infusion techniques for immunotherapy are known in the art (see, e.g., Rosenberg et al., New Eng. J. of Med. 319:1676 (1988)).

The individual, subject, or patient of the invention can be an animal, such as a mammal or a human.

As used herein, “treatment” or “treating” is an approach for obtaining beneficial or desired results, including clinical results. For purposes of this invention, beneficial or desired clinical results include but are not limited to one or more of the following: alleviating one or more symptoms resulting from a condition, diminishing the extent of a condition, stabilizing a condition (e.g., avoiding or delaying the worsening of the disease), delaying or slowing the progression of a condition, ameliorating a condition state, decreasing the dose of one or more other medications required to treat the condition, increasing the quality of life, and/or prolonging survival. Also encompassed by “treatment” is a reduction of a pathological consequence of a condition. The methods of the invention contemplate any one or more of these aspects of treatment.

The term “therapeutically effective amount” used herein refers to an amount of an agent, a combination of agents, or a pharmaceutical composition comprising such agents sufficient to treat a specified disorder, condition, or disease, such as to ameliorate, palliate, lessen, and/or delay one or more of its symptoms.

The term “alignment” refers to a method of comparing two or more polynucleotides or polypeptide sequences for the purpose of determining their relationship to each other. Alignments are typically performed by computer programs that apply various algorithms, however it is also possible to perform an alignment by hand. Alignment programs typically iterate through potential alignments of sequences and score the alignments using substitution tables, employing a variety of strategies to reach a potential optimal alignment score.

Commonly-used alignment algorithms include, but are not limited to, CLUSTALW, (scc, Thompson J. D., Higgins D. G., Gibson T. J., CLUSTAL W: improving the sensitivity of progressive multiple sequence alignment through sequence weighting, position-specific gap penalties and weight matrix choice, Nucleic Acids Research 22:4673-4680, 1994); CLUSTALV, (see, Larkin M. A., et al., CLUSTALW2, ClustalW and ClustalX version 2, Bioinformatics 23 (21): 2947-2948, 2007); Jotun-Hein, Muscle et al., MUSCLE: a multiple sequence alignment method with reduced time and space complexity, BMC Bioinformatics 5:113, 2004); Mafft, Kalign, ProbCons, and T-Coffee (see Notredame et al., T-Coffee: A novel method for multiple sequence alignments, Journal of Molecular Biology 302:205-217, 2000). Exemplary programs that implement one or more of the above algorithms include, but are not limited to MegAlign from DNAStar (DNAStar, Inc. 3801 Regent St. Madison, Wis. 53705), MUSCLE, T-Coffee, CLUSTALX, CLUSTALV, JalView, Phylip, and Discovery Studio from Accelrys (Accelrys, Inc., 10188 Telesis Ct, Suite 100, San Diego, Calif. 92121). In a non-limiting example, MegAlign is used to implement the CLUSTALW alignment algorithm with the following parameters: Gap Penalty 10, Gap Length Penalty 0.20, Delay Divergent Seqs (30%) DNA Transition Weight 0.50, Protein Weight matrix Gonnet Series, DNA Weight Matrix IUB.

The term “chromosomal integration” means the process whereby an incoming sequence is introduced into the chromosome of a host cell. The homologous regions of the transforming DNA align with homologous regions of the chromosome. Then, the sequence between the homology boxes can be replaced by the incoming sequence in a double crossover (i.e., homologous recombination). In some embodiments of the present invention, homologous sections of an inactivating chromosomal segment of a DNA construct align with the flanking homologous regions of the indigenous chromosomal region of the microbial chromosome. Subsequently, the indigenous chromosomal region is deleted by the DNA construct in a double crossover.

The term “consensus sequence” or “canonical sequence” refers to an archetypical amino acid sequence against which all variants of a particular protein or sequence of interest are compared. Either term also refers to a sequence that sets forth the nucleotides that are most often present in a polynucleotide sequence of interest. For each position of a protein, the consensus sequence gives the amino acid that is most abundant in that position in the sequence alignment.

The term “conservative substitutions” or “conserved substitutions” refers to, for example, a substitution of an amino acid with a conservative variant.

“Conservative variant” refers to residues that are functionally similar to a given residue. Amino acids within the following groups are conservative variants of one another: glycine, alanine, serine, and proline (very small); alanine, isoleucine, leucine, methionine, phenylalanine, valine, proline, and glycine (hydrophobic); alanine, valine, leucine, isoleucine, methionine (aliphatic-like); cysteine, serine, threonine, asparagine, tyrosine, and glutamine (polar); phenylalanine, tryptophan, tyrosine (aromatic); lysine, arginine, and histidine (basic); aspartate and glutamate (acidic); alanine and glycine; asparagine and glutamine; arginine and lysine; isoleucine, leucine, methionine, and valine; and serine and threoninc.

The terms “corresponds to,” “corresponding to,” or “aligning to” refer to an amino acid residue or position in a first protein sequence being positionally equivalent to an amino acid residue or position in a second reference protein sequence by virtue of the fact that the residue or position in the first protein sequence aligns to the residue or position in the reference sequence using bioinformatic techniques, for example, using the methods described herein for preparing a sequence alignment. The corresponding residue in the first protein sequence is then assigned the position number in the second reference protein sequence.

The term “deletion,” when used in the context of an amino acid sequence, means a deletion in or a removal of one or more residues from the amino acid sequence of a precursor protein, resulting in a mutant protein having at least one less amino acid residue as compared to the precursor protein. The term can also be used in the context of a nucleotide sequence, which means a deletion in or removal of a nucleotide from the polynucleotide sequence of a precursor polynucleotide.

The term “DNA construct” and “transforming DNA” (wherein “transforming” is used as an adjective) are used interchangeably herein to refer to a DNA used to introduce sequences into a host cell or organism. Typically a DNA construct is generated in vitro by PCR or other suitable technique(s) known to those in the art. In certain embodiments, the DNA construct comprises a sequence of interest (e.g., an incoming sequence). In some embodiments, the sequence is operably linked to additional elements such as control elements (e.g., promoters, etc.). A DNA construct can further comprise a selectable marker. It can also comprise an incoming sequence flanked by homology targeting sequences. In a further embodiment, the DNA construct comprises other non-homologous sequences, added to the ends (e.g., stuffer sequences or flanks). In some embodiments, the ends of the incoming sequence are closed such that the DNA construct forms a closed circle. The transforming sequences may be wildtype, mutant or modified. In some embodiments, the DNA construct comprises sequences homologous to the host cell chromosome. In other embodiments, the DNA construct comprises non-homologous sequences. Once the DNA construct is assembled in vitro it may be used to: 1) insert heterologous sequences into a desired target sequence of a host cell; 2) mutagenize a region of the host cell chromosome (i.e., replace an endogenous sequence with a heterologous sequence); 3) delete target genes; and/or (4) introduce a replicating plasmid into the host.

A polynucleotide is said to “encode” an RNA or a polypeptide if, in its native state or when manipulated by methods known to those of skill in the art, it can be transcribed and/or translated to produce the RNA, the polypeptide, or a fragment thereof. The antisense strand of such a polynucleotide is also said to encode the RNA or polypeptide sequences. As is known in the art, a DNA can be transcribed by an RNA polymerase to produce an RNA, and an RNA can be reverse transcribed by reverse transcriptase to produce a DNA. Thus, a DNA can encode an RNA, and vice versa.

The term “expressed genes” refers to genes that are transcribed into messenger RNA (mRNA) and then translated into protein, as well as genes that are transcribed into types of RNA, such as transfer RNA (tRNA), ribosomal RNA (rRNA), and regulatory RNA, which are not translated into protein.

The terms “expression cassette” or “expression vector” refer to a polynucleotide construct generated recombinantly or synthetically, with a series of specified elements that permit transcription of a particular polynucleotide in a target cell. A recombinant expression cassette can be incorporated into a plasmid, chromosome, mitochondrial DNA, plasmid DNA, virus, or polynucleotide fragment. Typically, the recombinant expression cassette portion of an expression vector includes, among other sequences, a polynucleotide sequence to be transcribed and a promoter. In particular embodiments, expression vectors have the ability to incorporate and express heterologous polynucleotide fragments in a host cell. Many prokaryotic and eukaryotic expression vectors are commercially available. Selection of appropriate expression vectors is within the knowledge of those of skill in the art. The term “expression cassette” is also used interchangeably herein with “DNA construct,” and their grammatical equivalents.

“Gene” refers to a polynucleotide (e.g., a DNA segment), which encodes a polypeptide, and may include regions preceding and following the coding regions as well as intervening sequences (introns) between individual coding segments (exons).

The term “endogenous protein” refers to a protein that is native to or naturally occurring in a cell. “Endogenous polynucleotide” refers to a polynucleotide that is in the cell and was not introduced into the cell using recombinant engineering techniques. For example, a gene that was present in the cell when the cell was originally isolated from nature. A gene is still considered endogenous if the control sequences, such as a promoter or enhancer sequences that activate transcription or translation, have been altered through recombinant techniques.

The term “homologous sequences” as used herein refers to a polynucleotide or polypeptide sequence having, for example, about 100%, about 99% or more, about 98% or more, about 97% or more, about 96% or more, about 95% or more, about 94% or more, about 93% or more, about 92% or more, about 91% or more, about 90% or more, about 88% or more, about 85% or more, about 80% or more, about 75% or more, about 70% or more, about 65% or more, about 60% or more, about 55% or more, about 50% or more, about 45% or more, or about 40% or more sequence identity to another polynucleotide or polypeptide sequence when optimally aligned for comparison. In particular embodiments, homologous sequences can retain the same type and/or level of a particular activity of interest. In some embodiments, homologous sequences have between 85% and 100% sequence identity, whereas in other embodiments there is between 90% and 100% sequence identity. In particular embodiments, there is 95% and 100% sequence identity.

“Homology” refers to sequence similarity or sequence identity. Homology is determined using standard techniques known in the art (see, e.g., Smith and Waterman, Adv. Appl. Math., 2:482, 1981; Needleman and Wunsch, J. Mol. Biol., 48:443, 1970; Pearson and Lipman, Proc. Natl. Acad. Sci. USA 85:2444, 1988; programs such as GAP, BESTFIT, FASTA, and TFASTA in the Wisconsin Genetics Software Package (Genetics Computer Group, Madison, Wis.); and Devereux et al., Nucl. Acid Res., 12:387-395, 1984). A non-limiting example includes the use of the BLAST program (Altschul et al., Gapped BLAST and PSI-BLAST: a new generation of protein database search programs, Nucleic Acids Res. 25:3389-3402, 1997) to identify sequences that can be said to be “homologous.” A recent version such as version 2.2.16, 2.2.17, 2.2.18, 2.2.19, or the latest version, including sub-programs such as blastp for protein-protein comparisons, blastn for nucleotide-nucleotide comparisons, tblastn for protein-nucleotide comparisons, or blastx for nucleotide-protein comparisons, and with parameters as follows: Maximum number of sequences returned 10,000 or 100,000; E-value (expectation value) of 1e-2 or 1e-5, word size 3, scoring matrix BLOSUM62, gap cost existence 11, gap cost extension 1, may be suitable. An E-value of 1e-5, for example, indicates that the chance of a homologous match occurring at random is about 1 in 10,000, thereby marking a high confidence of true homology.

The term “identical” (or “identity”), in the context of two polynucleotide or polypeptide sequences, means that the residues in the two sequences are the same when aligned for maximum correspondence, as measured using a sequence comparison or analysis algorithm such as those described herein. For example, if when properly aligned, the corresponding segments of two sequences have identical residues at 5 positions out of 10, it is said that the two sequences have a 50% identity. Most bioinformatic programs report percent identity over aligned sequence regions, which are typically not the entire molecules. If an alignment is long enough and contains enough identical residues, an expectation value can be calculated, which indicates that the level of identity in the alignment is unlikely to occur by random chance.

The term “introduce” in the context of introducing the degradation targeting agent in a particular location (e.g., a cell, body, subject, patient, individual) refers to causing the appearance of the degradation targeting agent in that location, such as by expressing the degradation targeting agent via a nucleic acid in the location or delivering a pre-generated degradation targeting agent to the location.

The term “isolated” or “purified” means a material that is removed from its original environment, for example, a host cell if it is produced in a host cell, the natural environment if it is naturally occurring, or a cultivation broth if it is produced in a recombinant host cell cultivation medium. A material is said to be “purified” when it is present in a particular composition in a higher concentration than the concentration that exists prior to the purification step(s). For example, with respect to an element normally found in a cell, organism, or microbe (including a phage), such an element is “purified” when the final composition does not include some material from the original matrix.

The term “mutation” refers to, in the context of a polynucleotide, a modification to the polynucleotide sequence resulting in a change in the sequence of a polynucleotide with reference to a precursor polynucleotide sequence. A mutant polynucleotide sequence can refer to an alteration that does not change the encoded amino acid sequence, for example, with regard to codon optimization for expression purposes, or that modifies a codon in such a way as to result in a modification of the encoded amino acid sequence. Mutations can be introduced into a polynucleotide through any number of methods known to those of ordinary skill in the art, including random mutagenesis, site-specific mutagenesis, oligonucleotide directed mutagenesis, gene shuffling, directed evolution techniques, combinatorial mutagenesis, site saturation mutagenesis among others.

“Mutation” or “mutated” means, in the context of a protein, a modification to the amino acid sequence resulting in a change in the sequence of a protein with reference to a precursor protein sequence. A mutation can refer to a substitution of one amino acid with another amino acid, an insertion or a deletion of one or more amino acid residues. Specifically, a mutation can also be the replacement of an amino acid with a non-natural amino acid, or with a chemically-modified amino acid or like residues. A mutation can also be a truncation (e.g., a deletion or interruption) in a sequence or a subsequence from the precursor sequence. A mutation may also be an addition of a subsequence (e.g., two or more amino acids in a stretch, which are inserted between two contiguous amino acids in a precursor protein sequence) within a protein, or at either terminal end of a protein, thereby increasing the length of (or elongating) the protein. A mutation can be made by modifying the DNA sequence corresponding to the precursor protein. Mutations can be introduced into a protein sequence by known methods in the art, for example, by creating synthetic DNA sequences that encode the mutation with reference to precursor proteins, or chemically altering the protein itself. A “mutant” as used herein is a protein comprising a mutation.

The term “operably linked,” in the context of a polynucleotide sequence, refers to the placement of one polynucleotide sequence into a functional relationship with another polynucleotide sequence. For example, a DNA encoding a secretory leader (e.g., a signal peptide) is operably linked to a DNA encoding a polypeptide if it is expressed as a preprotein that participates in the secretion of the polypeptide. A promoter or an enhancer is operably linked to a coding sequence if it affects the transcription of the sequence. A ribosome binding site is operably linked to a coding sequence if it is positioned so as to facilitate translation. Generally, “operably linked” means that the DNA sequences being linked are contiguous, and, in the case of a secretory leader, contiguous and in the same reading frame.

The term “optimal alignment” refers to the alignment giving the highest overall alignment score.

The terms “percent sequence identity,” “percent amino acid sequence identity,” “percent gene sequence identity,” and/or “percent polynucleotide sequence identity,” with respect to two polypeptides, polynucleotides and/or gene sequences (as appropriate), refer to the percentage of residues that are identical in the two sequences when the sequences are optimally aligned. Thus, 80% amino acid sequence identity means that 80% of the amino acids in two optimally aligned polypeptide sequences are identical. The percent identities expressed herein with respect to a given named reference sequence are determined over the entire reference sequence, rather than only a portion thereof. Thus, an amino acid sequence at least about 80% identical to positions 1-42 of SEQ ID NO:4, for example, is at least about 80% identical to the entire sequence of positions 1-42 of SEQ ID NO:4, as opposed merely to subsequences thereof.

The term “plasmid” refers to a circular double-stranded (ds) DNA construct used as a cloning vector, and which forms an extrachromosomal self-replicating genetic element in some eukaryotes or prokaryotes, or integrates into the host chromosome.

A “promoter” is a polynucleotide sequence that functions to direct transcription of a downstream gene. In preferred embodiments, the promoter is appropriate to the host cell in which the target gene is being expressed. The promoter, together with other transcriptional and translational regulatory polynucleotide sequences (also termed “control sequences”) is necessary to express a given gene. In general, the transcriptional and translational regulatory sequences include, but are not limited to, promoter sequences, ribosomal binding sites, transcriptional start and stop sequences, translational start and stop sequences, and enhancer or activator sequences.

“Recombinant”: A recombinant nucleic acid or polypeptide is one comprising a sequence that is not naturally occurring. A recombinant gene is a gene that comprises a recombinant nucleic acid sequence, is present within a cell in which it does not naturally occur, and/or is present in a different locus (e.g., genetic locus or on an extrachromosomal plasmid) within a particular cell than in a corresponding native cell. A recombinant cell (such as a recombinant microorganism) is one that comprises a recombinant nucleic acid, a recombinant gene, or a recombinant polypeptide. An example of a recombinant gene is a gene that has a coding sequence operably linked to a heterologous promoter. The degradation targeting agents of the invention or the nucleic acids configured to express same can be recombinant.

The terms “regulatory segment,” “regulatory sequence,” or “expression control sequence” refer to a polynucleotide sequence that is operatively linked with another polynucleotide sequence that encodes the amino acid sequence of a polypeptide chain to effect the expression of that encoded amino acid sequence. The regulatory sequence can inhibit, repress, promote, or even drive the expression of the operably-linked polynucleotide sequence encoding the amino acid sequence.

The term “substantially identical,” in the context of two polynucleotides or two polypeptides refers to a polynucleotide or polypeptide that comprises at least 70% sequence identity, for example, at least 75%, at least 80%, at least 85%, at least 90%, at least 91%, at least 92%, at least 93%, at least 94%, at least 95%, at least 96%, at least 97%, at least 98%, or at least 99% sequence identity as compared to a reference sequence using the programs or algorithms (e.g., BLAST, ALIGN, CLUSTAL) using standard parameters.

“Substantially purified” means molecules that are at least about 60% free, preferably at least about 75% free, about 80% free, about 85% free, and more preferably at least about 90% free from other components with which they are naturally associated. As used herein, the term “purified” or “to purify” also refers to the removal of contaminants from a sample.

“Substitution” means replacing an amino acid in the sequence of a precursor protein with another amino acid at a particular position, resulting in a mutant of the precursor protein. The amino acid used as a substitute can be a naturally-occurring amino acid, or can be a synthetic or non-naturally-occurring amino acid.

The term “transformed” or “stably transformed” cell refers to a cell that has a non-native (heterologous) polynucleotide sequence integrated into its genome or as an episomal plasmid that is maintained for at least two generations.

“Vector” refers to a polynucleotide construct designed to introduce polynucleotides into one or more cell types. Vectors include cloning vectors, expression vectors, shuttle vectors, plasmids, cassettes and the like.

The elements and method steps described herein can be used in any combination whether explicitly described or not.

All combinations of method steps as used herein can be performed in any order, unless otherwise specified or clearly implied to the contrary by the context in which the referenced combination is made.

As used herein, the singular forms “a,” “an,” and “the” include plural referents unless the content clearly dictates otherwise.

Numerical ranges as used herein are intended to include every number and subset of numbers contained within that range, whether specifically disclosed or not. Further, these numerical ranges should be construed as providing support for a claim directed to any number or subset of numbers in that range. For example, a disclosure of from 1 to 10 should be construed as supporting a range of from 2 to 8, from 3 to 7, from 5 to 6, from 1 to 9, from 3.6 to 4.6, from 3.5 to 9.9, and so forth.

All patents, patent publications, and peer-reviewed publications (i.e., “references”) cited herein are expressly incorporated by reference to the same extent as if each individual reference were specifically and individually indicated as being incorporated by reference. In case of conflict between the present disclosure and the incorporated references, the present disclosure controls.

It is understood that the invention is not confined to the particular construction and arrangement of parts herein illustrated and described, but embraces such modified forms thereof as come within the scope of the claims.

### EXAMPLES

### Results

### HLTF;zf-C3HC4 and RO52;zf-C3HC4 Have Targeted Protein Degradation Capabilities

While screening for peptides that affect genome editing, we identified a peptide, named HLTF;zf-C3HC4 (nomenclature: gene; domain) (SEQ ID NO:2, encoded by SEQ ID NO:1), that potentially caused targeted degradation of Cas9. To test the degradation ability of this peptide, we generated two lentiviral plasmids. A first included a construct (Ef1a-Cas9-ABI-T2A-GFP-P2A-Blast (SEQ ID NO:25)) that expressed Cas9 fused to a Flag tag, an ABI domain and a nuclear localization signal (NLS) (referred to as “Cas9-ABI”) and included a green fluorescent protein (GFP) marker and blasticidin resistance sequence downstream. A second included a construct (Ef1a-HA-PYL1-NLS-HLTF/RO52;zfC3HC4-T2A-mCherry-P2A-Puro (SEQ ID NO:27) with SEQ ID NO:1 in the placeholder sequence thereof) that expressed HLTF;zf-C3HC4 (SEQ ID NO:2) fused to an HA tag, a PYL1 domain (an abscisic acid receptor), and an NLS (referred to as “PYL1-HLTF;zf-C3HC4”) and included a mCherry marker and a puromycin resistance sequence downstream. ABI and PYL1 are domains that dimerize in the presence of abscisic acid (ABA) (Miyazono K, Miyakawa T, Sawano Y, Kubota K, Kang HJ, Asano A, Miyauchi Y, Takahashi M, Zhi Y, Fujita Y, Yoshida T, Kodaira KS, Yamaguchi-Shinozaki K, Tanokura M. Structural basis of abscisic acid signalling. Nature. 2009 Dec. 3; 462 (7273): 609-14). These dimerizing domains allowed inducible recruitment of PYL1-HLTF;zf-C3HC4 as an exemplary degradation targeting agent to Cas9-ABI as an exemplary target molecule, wherein the PYL1 domain in PYL1-HLTF;zf-C3HC4 (and other analogous fusions described in further detail below) served as an exemplary ligand moiety and HLTF;zf-C3HC4 served as an exemplary degradation targeting peptide.

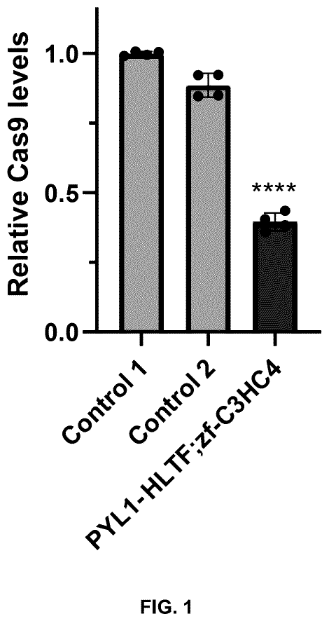

K562 cells were sequentially transduced with lentiviruses for expression of the Cas9-ABI and PYL1-HLTF;zf-C3HC4 peptides. Negative control versions of this cell line were made using control peptides comprising sequences from the DMD gene. Cells were treated with ABA and stained for levels of Cas9 (as Cas9-ABI). We observed reduced Cas9 protein levels in ABA-treated cells expressing PYL1-HLTF;zf-C3HC4 compared to Cas9 levels in the ABA-treated control cell lines (FIG. 1).

Having shown that HLTF;zf-C3HC4 can reduce Cas9 protein levels when recruited in close proximity, we searched for additional peptides in our screen that may have targeted degradation ability. HLTF is an E3 ligase, which is a family of proteins known for ubiquitinating substrates to direct them for degradation. The zf-C3HC4 domain encoded in the peptide is responsible for HLTF's ubiquitination activity. We hypothesized that other peptides that encoded a zf-C3HC4 domain and reduced Cas9 editing efficiency in our screen for peptides that affect genome editing, which was the phenotype that brought HLTF;zf-C3HC4 to our attention, might have degradation capabilities. RO52;zf-C3HC4 (SEQ ID NO:16, encoded by SEQ ID NO:15) showed a significant decrease in Cas9 editing efficiency in our screen, so we included the peptide in further degradation assays analogous to those described above by expressing RO52;zf-C3HC4 (SEQ ID NO:16) fused to an HA tag, a PYL1 domain, and an NLS (referred to as “PYL1-RO52;zf-C3HC4”) from the Ef1a-HA-PYL1-NLS-HLTF/RO52;zfC3HC4-T2A-mCherry-P2A-Puro (SEQ ID NO:27) construct with SEQ ID NO: 15 in the placeholder sequence thereof. Our results (data not shown) indicated that PYL1-RO52;zf-C3HC4 could induce degradation of Cas9-ABI.

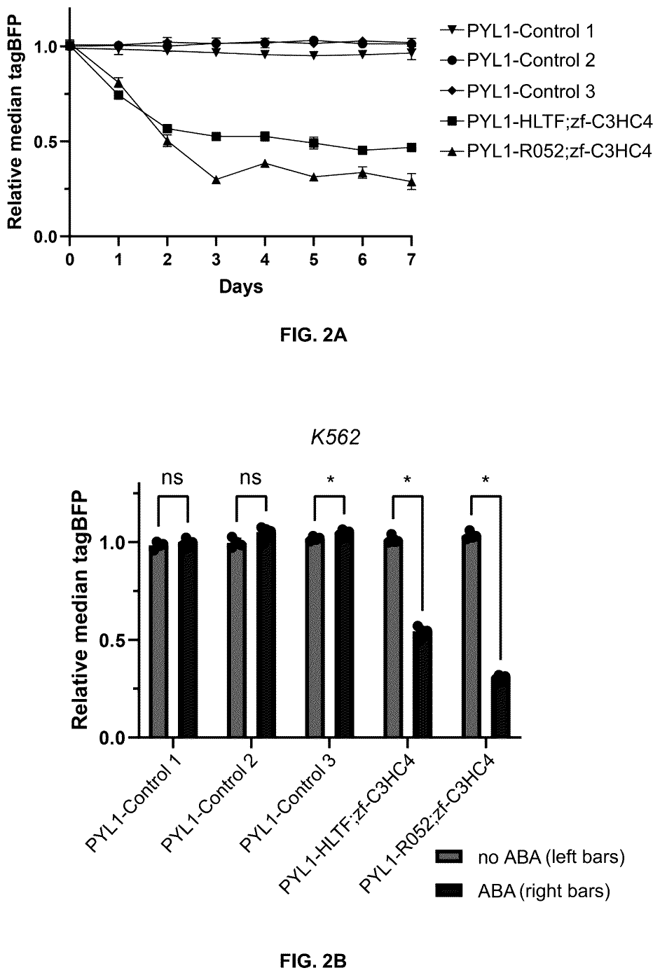

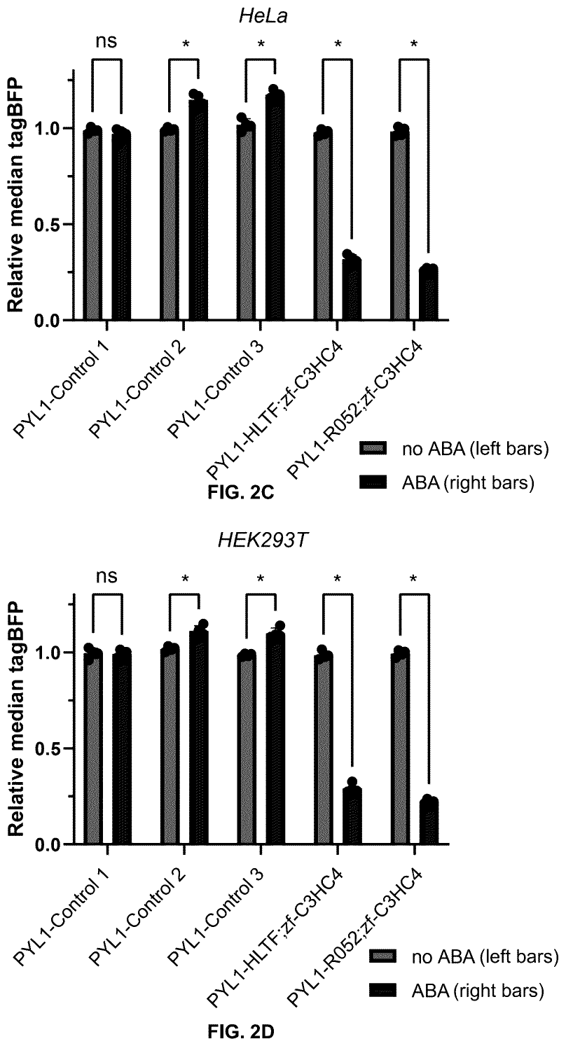

Our initial screening data and degradation assay results suggested HLTF;zf-C3HC4 and RO52;zf-C3HC4 could induce degradation of Cas9, but we wanted to determine whether their degradation activity was specific to Cas9. We therefore tested whether these peptides could degrade tagBFP fused to the ABI domain and an NLS (“tagBFP-ABI”). We established K562 cells comprising a construct (Ef1a-tagBFP-ABI-T2A-GFP-P2A-Blast, SEQ ID NO:26) expressing the tagBFP-ABI fusion. We then expressed PYL1-HLTF;zf-C3HC4, PYL1-RO52;zf-C3HC4, or three control fusions (PYL1-Control 1, PYL1-Control 2, and PYL1-Control 3; expressed from the Ef1a-HA-PYL1-NLS-HLTF/RO52;zfC3HC4-T2A-mCherry-P2A-Puro (SEQ ID NO:27) construct with control sequences in the placeholder sequence of the Ef1a-HA-PYL1-NLS-HLTF/RO52;zfC3HC4-T2A-mCherry-P2A-Puro construct) in the cells. Cells expressing both PYL1-HLTF;zf-C3HC4 and PYL1-RO52;zf-C3HC4 exhibited reduced median tagBFP fluorescence, reaching a maximum reduction in fluorescence after three days (FIG. 2A). Thus, all further degradation assay measurements were taken three days after ABA treatment unless otherwise specified. PYL1-RO52;zf-C3HC4 more efficiently reduced tagBFP levels (72%) compared to PYL1-HLTF;zf-C3HC4 (46%) (FIG. 2B). In both cases, this degradation required the presence of ABA showing that inducing proximity between the peptide and target is necessary for degradation. The control fusions (PYL1-Control 1, PYL1-Control 2, and PYL1-Control 3) did not exhibit a significant reduction in tagBFP levels when ABA was added, except for PYL1-Control 3, which increased the relative median tagBFP signal by 3%. We conducted the tagBFP degradation assay in HEK293T and HeLa cells and observed ABA-dependent degradation of tagBFP for both zf-C3HC4 peptides in both of these cell lines. Similarly, as in K562 cells, PYL1-Control 2 and PYL1-Control 3 increased the relative median tagBFP signal by 10% in HEK293T cells (FIG. 2D) and 24% in HeLa cells (FIG. 2C). These results suggest that the targeted degradation abilities are not specific to K562 cells.

### Effects of the E2-Binding Region, Zinc-Binding Region, and C-Terminus of HLTF;<f-C3HC4

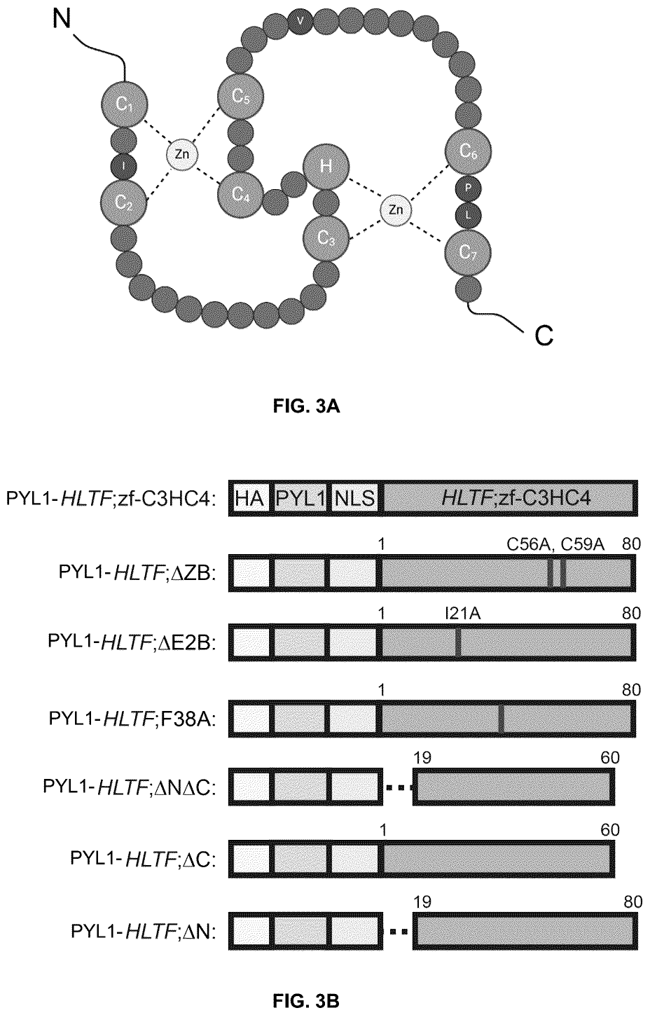

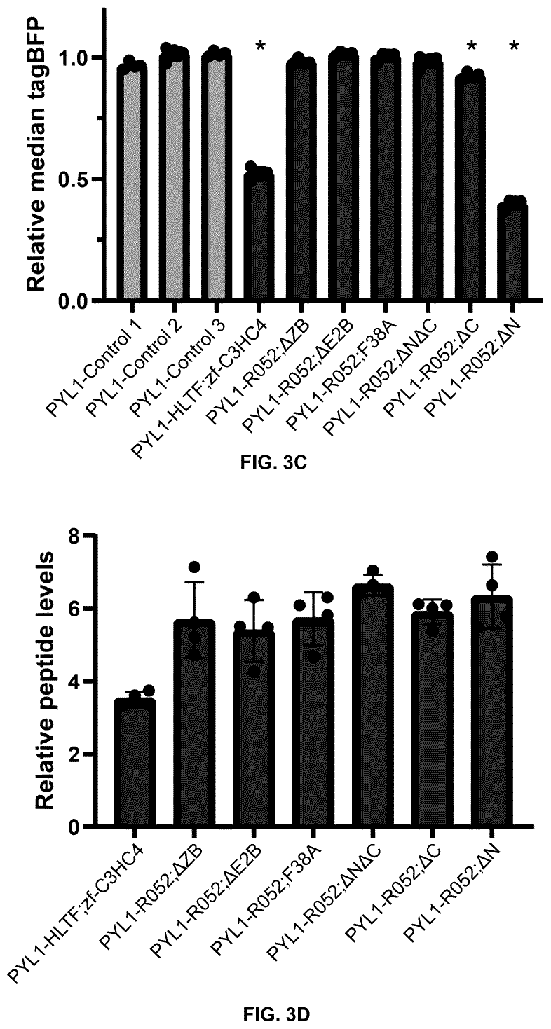

We wanted to determine which components of the HLTF;zf-C3HC4 peptide (SEQ ID NO: 2) were necessary for targeted degradation. The HLTF;zf-C3HC4 peptide contains a RING domain (SEQ ID NO:8), which contains an E2-binding and zinc-binding regions that are essential for its ubiquitination function (FIG. 3A). HLTF;zf-C3HC34 also contains an additional 18 (at the N-terminus) and 20 (at the C-terminus) amino acids of HLTF gene sequence on either side of the RING domain. We generated HLTF;zf-C3HC34 truncations that removed the N-terminus of the peptide (HLTF;ΔN) (SEQ ID NO:6, encoded by SEQ ID NO: 5), the C-terminus of the peptide (HLTF;ΔC) (SEQ ID NO:4, encoded by SEQ ID NO:3), or both (HLTF;ΔNΔC) (SEQ ID NO:8, encoded by SEQ ID NO:7). We also generated HLTF;zf-C3HC4 mutants with mutations to the RING domain that disrupt the E2-binding (HLTF;ΔE2B, containing an I21A substitution) (SEQ ID NO:10, encoded by SEQ ID NO:9) or zinc-binding functions (HLTF;ΔZB, containing C56A and C59A substitutions) (SEQ ID NO: 14, encoded by SEQ ID NO:13). Finally, we generated a HLTF;zf-C3HC4 mutant (HLTF;F38A) (SEQ ID NO:12, encoded by SEQ ID NO:11) with a substitution at a phenylalanine that was conserved across zf-C3HC4 RING domains. We tested these mutants for tagBFP-ABI degradation by expressing these peptides as PYL1 fusions (“PYL1-[Mutant Abbreviation]”) (FIG. 3B) from the Ef1a-HA-PYL1-NLS-HLTF/RO52;zfC3HC4-T2A-mCherry-P2A-Puro (SEQ ID NO:27) construct with the various coding sequences of the HLTF;zf-C3HC34 mutants included in the Ef1a-HA-PYL1-NLS-HLTF/RO52;zfC3HC4-T2A-mCherry-P2A-Puro placeholder sequence.

As shown in FIG. 3C, none of the point mutants (PYL1-HLTF;ΔZB, PYL1-HLTF;ΔE2B, PYL1-HLTF;F38A) showed significant degradation activity of tagBFP, nor did the ΔNΔC truncation (PYL1-HLTF;ΔNΔC). The ΔC truncation (PYL1-HLTF;ΔC) severely reduced degradation activity, only degrading protein levels to 93% of control levels, and the ΔN truncation (PYL1-HLTF;ΔN) showed a slight enhancement of degradation compared to the full peptide (PYL1-HLTF;zf-C3HC4), degrading protein levels to 40% as opposed to 52% in the full peptide (FIG. 3C). We stained cells expressing the HLTF;zf-C3HC4 mutants for the protein level of the peptide via the HA-tag fused to the PYL1-peptide construct to determine if these mutants had altered protein levels that could explain the loss of degradation activity (FIG. 3D). We found no reduction in peptide levels for the mutants but instead detected an increase in mutant peptide levels. Given the E2 domain and zinc-binding regions were necessary for degradation function, we concluded that the ability to ubiquitinate is essential for HLTF;zf-C3HC4 to degrade its target protein.

### Effect of NLS

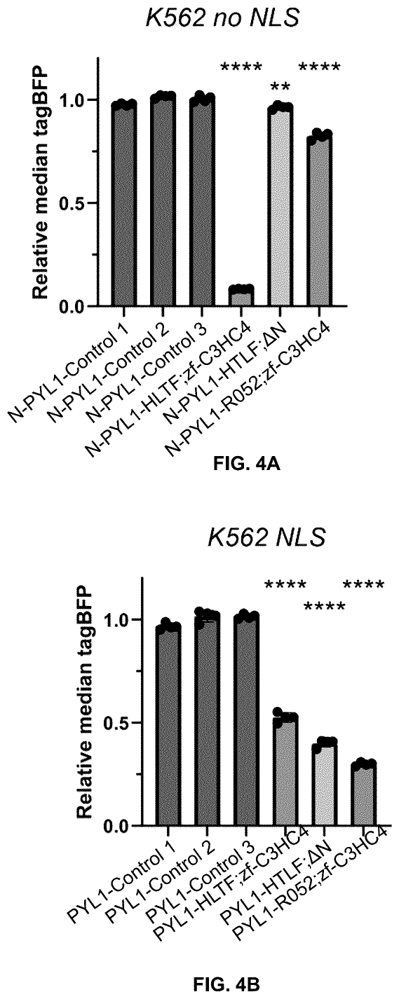

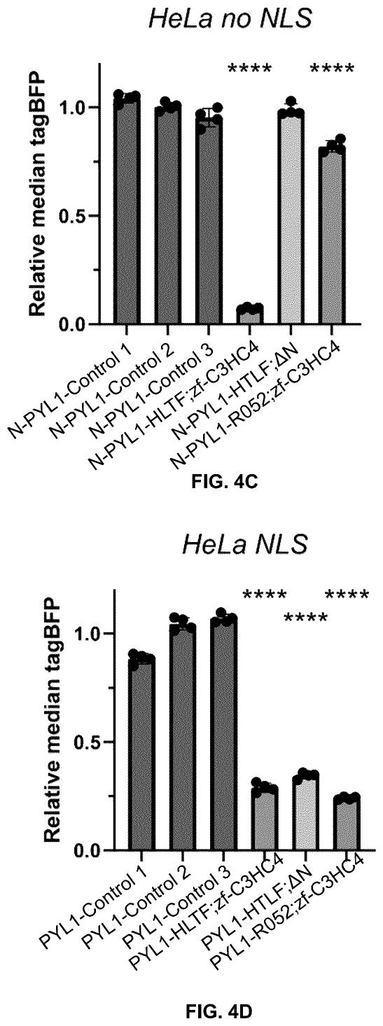

In the Cas9 and tagBFP degradation assays, both the expressed peptide and targeted substrates contained an NLS. Therefore, we tested whether HLTF;zf-C3HC4 and RO52;zf-C3HC4 could degrade targets outside of the nucleus. To test this possibility, we generated expression vectors without an NLS for the peptide and tagBFP vectors (SEQ ID NO:28). We tested both zf-C3HC4 peptides in a degradation assay using these no-NLS constructs (FIGS. 4A-4D) (referred to as (“N-PYL1-[zf-C3HC4 Domain Abbreviation]”) in both K562 and HeLa cells. We observed enhanced degradation with N-PYL1-HLTF;zf-C3HC4 relative to PYL1-HLTF;zf-C3HC4 (FIGS. 4A-4D). However, the loss of the NLS in N-PYL1-RO52;zf-C3HC4 and N-PYL1-HLTF;ΔN relative to PYL1-RO52;zf-C3HC4 and PYL1-HLTF;ΔN, respectively, resulted in decreased degradation activity (FIGS. 4A-4D). These results show that HLTF;zf-C3HC4 is capable of inducing degradation of substrates outside of the nucleus but that RO52;zf-C3HC4 and HLTF;ΔN, while technically capable of degradation in the cytosol, have attenuated activity outside the nucleus.

### Tests for Proteolytic Cleavage

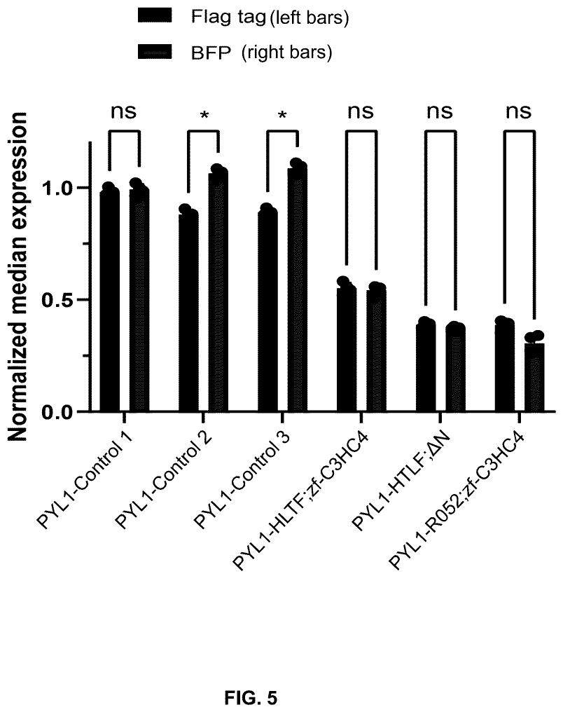

We wanted to explore the mechanism by which these peptides were inducing degradation of proteins. We hypothesized that HLTF;zf-C3HC4 and RO52;zf-C3HC4 could induce loss of tagBFP by either proteolysis or complete degradation. If the former were true, small fragments that include the Flag tag fused to tagBFP-ABI should be present even when tagBFP fluorescence was reduced. We stained cells expressing PYL1-zf-C3HC4 fusion peptides with tagBFP-ABI, with and without ABA, and tagBFP signal was measured via flow cytometry. The levels of Flag tag were reduced by a similar amount as tagBFP fluorescence in cells expressing the PYL1-zf-C3HC4 fusion peptides and PYL1-Control 1. PYL1-Control 2 and PYL1-Control 3 showed a respective increase of 18 and 19% of tagBFP levels compared to Flag tag (FIG. 5). This finding supports that tagBFP is not being proteolytically cleaved.

### Tests for Ubiquitin-Dependence of Targeted Degradation by HLTF;zf-C3CH4 and RO52;zf-C3HC4

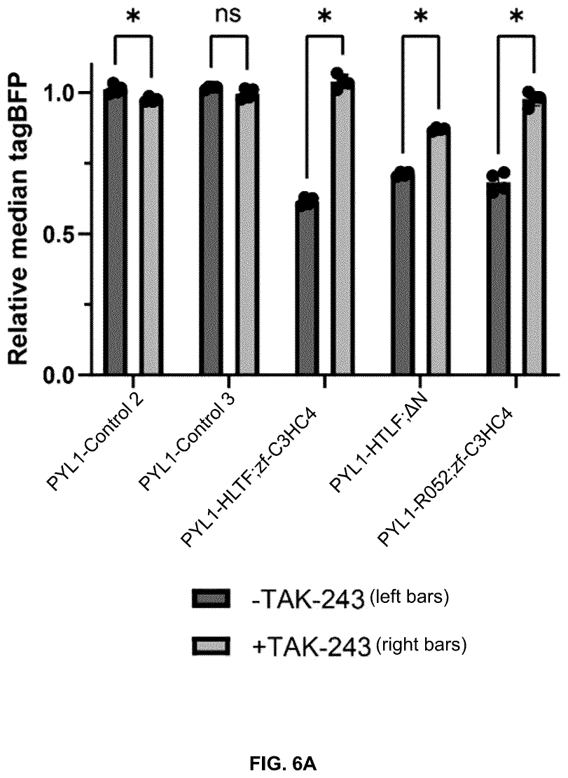

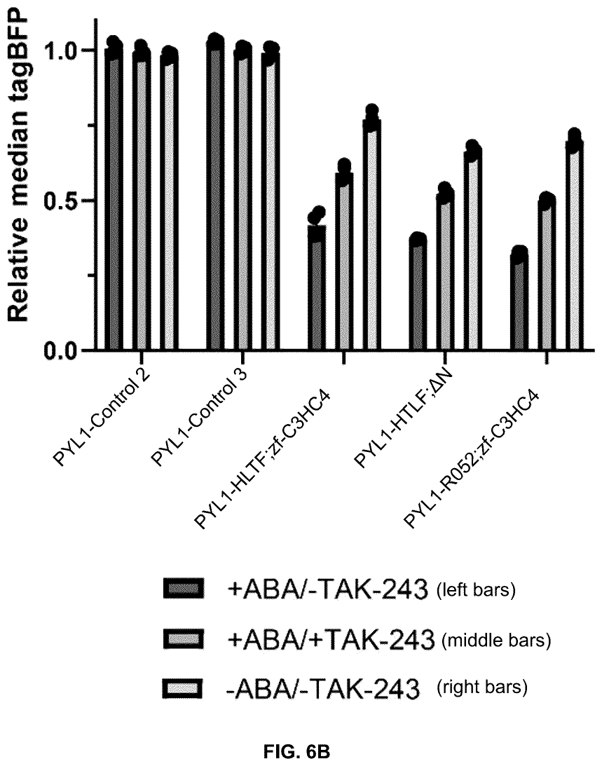

RING domains contain an E2-binding domain and zinc-binding regions that are responsible for ubiquitination and which we identified as essential to the functioning of HLTF;zf-C3HC4 and RO52;zf-C3HC4. To confirm that the peptides' function is ubiquitination-dependent, we treated the peptide-expressing cells with TAK-243, a ubiquitin inhibitor. In one experiment, we co-treated the peptide-expressing cells for 20 hours with ABA to induce degradation and TAK-243 to inhibit degradation (FIG. 6A) and found that degradation activity was significantly inhibited in TAK-243 treated cells. In a similar experiment, we first treated peptide-expressing cells with ABA for three days to reach the maximum amount of degradation, then treated the cells with TAK-243 or removed ABA to compare the amount of TAK-243 rescue to natural recovery of BFP expression. While cells treated with both TAK-243 and continuous ABA did not reach the same levels of BFP as cells with ABA removed, they did show significant recovery compared to cells untreated with TAK-243 (FIG. 6B).

### Tests for Proteasome-Dependence of Targeted Degradation by HLTF;zf-C3HC4 and RO52;zf-C3HC4

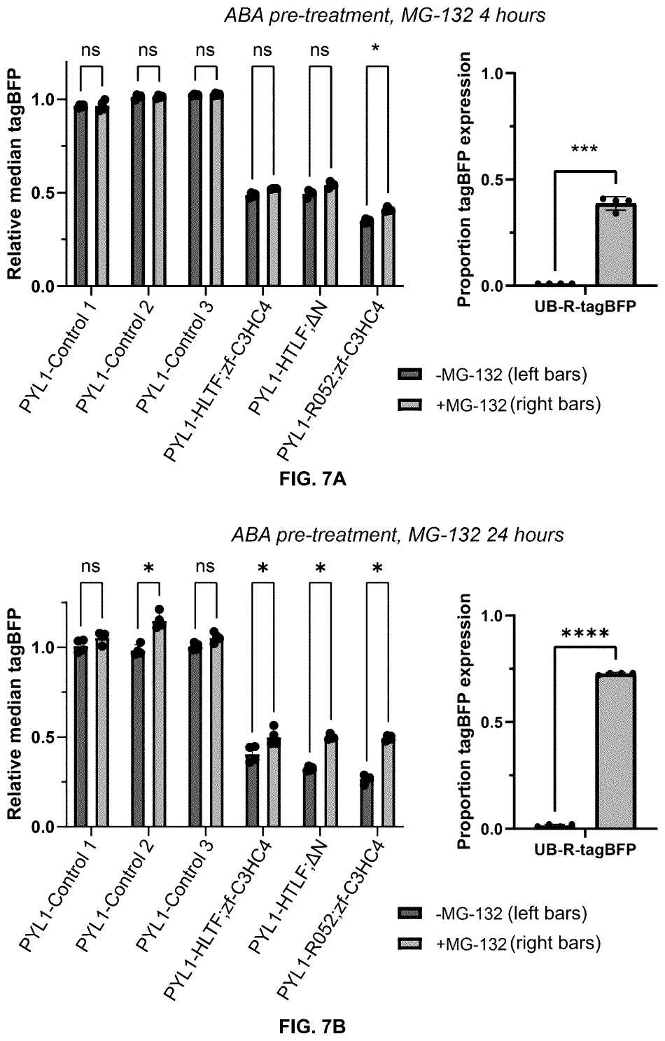

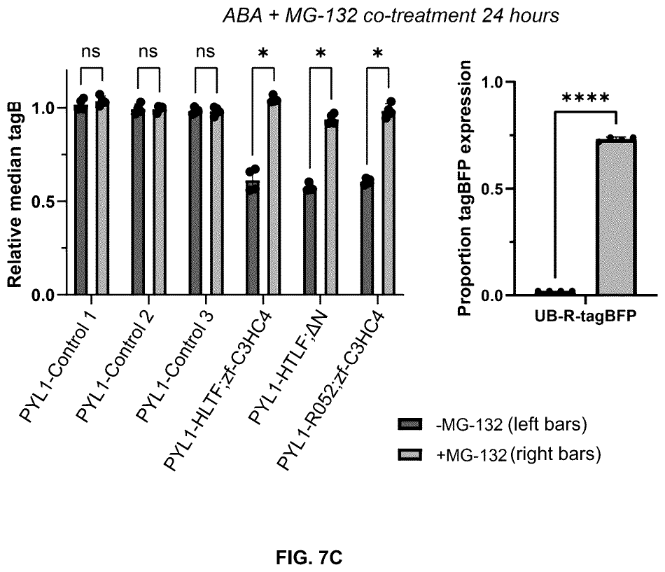

There are two main pathways for complete protein degradation, the proteasome, which degrades cytosolic proteins, and the lysosome, which can degrade cytosolic, membrane, and extracellular proteins as well as organelles. To determine if the zf-C3HC4 domains are using the proteasome to degrade their targets, we treated the ABA-treated cells expressing the PYL1-HLTF;zf-C3HC4 and PYL1-RO52;zf-C3HC4 peptides with MG-132, a proteasome inhibitor, and measured median tagBFP levels with and without MG-132. If tagBFP degradation is dependent on the proteasome, we would expect to see an increase in tagBFP fluorescence in the presence of MG-132. To confirm successful inhibition of the proteasome by MG-132, we used a reporter that expresses tagBFP fused to ubiquitin (UB-R-tagBFP) (Dantuma NP, Lindsten K, Glas R, Jellne M, Masucci MG. Short-lived green fluorescent proteins for quantifying ubiquitin/proteasome-dependent proteolysis in living cells. Nat Biotechnol. 2000 May; 18 (5): 538-43) and confirmed that treatment with MG-132 rescued tagBFP fluorescence (FIGS. 7A-7C, right panels). Only PYL1-RO52;zf-C3HC4 showed a significant increase in tagBFP levels after treatment with MG-132 for four hours, although the peptide still efficiently reduced tagBFP expression (41% of control levels) (FIG. 7A, left panel). We repeated the test with a 24-hour incubation or by treating the cells with ABA and MG-132 at the same time. Treating peptide-expressing cells with MG-132 for 24 hours after degradation had reached its maximum point resulted in all three peptides showed a significant increase in tagBFP levels, though as before, they still efficiently reduced expression of tagBFP (50% or less of control levels) (FIG. 7B left panel). However, co-treatment of cells with ABA and MG-132 with a 24-hour incubation effectively prevented degradation (FIG. 7C, left panel). These results suggest that HLTF;zf-C3HC4 and RO52;zf-C3HC4 are at least partially proteasome-dependent.

### Tests for Lysosome-Dependence of Targeted Degradation by HLTF;zf-C3HC4 and RO52;zf-C3HC4

Another major degradation pathway is the lysosome. To test for lysosome-dependence of the targeted degradation by HLTF;zf-C3HC4 and RO52;zf-C3HC4, CRISPR knockout lines of a key lysosomal degradation gene, atg5, can be generated and the peptide and tagBFP constructs can be co-expressed in these cell lines. If degradation is dependent on the lysosome, we would expect to see an increase in tagBFP.

### Materials and Methods

tagBFP Degradation Assay

Cell lines were transduced with the target protein virus (Cas9-ABI or tagBFP-ABI) and selected with blasticidin for seven days. Once this cell line was established, it was transduced with lentivirus carrying the peptide expression cassette in two replicates. The peptide infection remained unselected in order to maintain a mixed population of peptide-positive and peptide-negative cells, which could be distinguished using the mCherry marker on the peptide expression vector. The cells were plated in 24-well plates. Assays using K562 cells were plated at 500,000 cells in 2 mL of media. Assays using HEK293T and HeLa cells were plated at 125,000 cells in 1 mL of media. Each cell line was plated in four replicates with two replicates from each replicate infection. Cells were treated with ABA (100 M) for three days and analyzed via flow cytometry. The median tagBFP levels were first normalized within each well by dividing the median of the cell expressing a peptide (mCherry+) by the median of cells not expressing a peptide (mCherry-). Next, each well was normalized to the average of the non-ABA control peptide wells or ABA control peptide wells.

### Proteasomal Dependence Assay

Cells were plated and treated as described in the degradation assay. After three days of treatment with ABA, the cells were divided into two wells, and one well was treated with 5 μM MG132 while the other was left untreated. The cells were incubated for either 4 or 24 hours and measured by flow cytometry. In co-treatment, cells were treated with ABA and MG-132 at the same time and incubated for 24 hours before measurement by flow cytometry. The median tagBFP level was first normalized to the peptide-negative (mCherry-) population within each well as described above. Next, the normalized median tagBFP of each treated well was compared to its untreated counterpart using a paired T-test.

### Ubiquitination-Dependence Assay

Cells were plated and treated as described in the degradation assay. After three days of treatment with ABA, the cells were divided into three wells. One well was treated with 500 nM TAK-243, one was kept under ABA, and one had ABA removed. The cells were incubated for 18 hours, and measured by flow cytometry. In the co-treatment assay, cells were treated with ABA and TAK-243 at the same time and incubated for 20 hours before measurement by flow cytometry. The median tagBFP level was first normalized to the peptide-negative (mCherry-) population within each well as described above. Next, the normalized median tagBFP of each treated well was compared to its untreated counterpart using a paired T-test.

### Intracellular Staining for Cas9, HA-Tag, and Flag-Tag

A mixture of cells expressing the peptide or not expressing the peptide was treated for three days with ABA. Each cell line was treated in four replicates with two replicates from each infection replicate. On the third day, 1 million cells were collected from each replicate. They were fixed in Fix Buffer I (BD) at 37° C. for 15 minutes and washed with PBS with 10% FBS. They were permeabilized with Perm Buffer III (BD) on ice for 30 minutes and washed again. They were incubated with primary antibody diluted in wash buffer at room temperature for one hour, washed and incubated with secondary antibody diluted in wash buffer at 4° C. for one hour in the dark. They were washed, resuspended in wash buffer, and measured via flow cytometry. The Cas9 staining used anti-Cas9 (Thermofisher Scientific 7A9-3A3, 1:500). The staining of peptide levels used anti-HA (Cell Signaling Technology 6E2, 1:50). The staining of the Flag epitope used anti-Flag (Millipore Sigma F1804, 1:500 dilution). All stainings used an Alexa-647 anti-mouse secondary antibody (Fisher Scientific A-21235, 1:1000).

SEQUENCES

SEQ ID NO: 1
HLTF; zf-C3HC4 Coding Sequence
AAGAAGCTGATCAGAAAGATGAAGCTGATCCTGAGCAGCGGCAGCGACGAGGA
GTGCGCCATCTGCCTGGACAGCCTGACCGTGCCCGTGATCACCCACTGCGCCCA
CGTGTTCTGCAAGCCCTGCATCTGCCAGGTGATCCAGAACGAGCAGCCCCACGC
CAAGTGCCCCCTGTGCAGAAACGACATCCACGAGGACAACCTGCTGGAGTGCCC
CCCCGAGGAGCTGGCCAGAGACAGC
SEQ ID NO: 2
HLTF; zf-C3HC4 Protein
KKLIRKMKLILSSGSDEECAICLDSLTVPVITHCAHVFCKPCICQVIQNEQPHAKCP
LCRNDIHEDNLLECPPEELARDS
Bold: N- and C-termini
I21A: E2-binding domain dead mutation
F38A: Conserved residue F > A point mutation
C56A and C59A: Ligase domain dead mutation
SEQ ID NO: 3
HLTF; ΔC Coding Sequence
AAGAAGCTGATCAGAAAGATGAAGCTGATCCTGAGCAGCGGCAGCGACGAGGA
GTGCGCCATCTGCCTGGACAGCCTGACCGTGCCCGTGATCACCCACTGCGCCCA
CGTGTTCTGCAAGCCCTGCATCTGCCAGGTGATCCAGAACGAGCAGCCCCACGC
CAAGTGCCCCCTGTGCAGA
SEQ ID NO: 4
HLTF; ΔC Protein
KKLIRKMKLILSSGSDEECAICLDSLTVPVITHCAHVFCKPCICQVIQNEQPHAKCP
LCR
SEQ ID NO: 5
HLTF; ΔN Coding Sequence
TGCGCCATCTGCCTGGACAGCCTGACCGTGCCCGTGATCACCCACTGCGCCCAC
GTGTTCTGCAAGCCCTGCATCTGCCAGGTGATCCAGAACGAGCAGCCCCACGCC
AAGTGCCCCCTGTGCAGAAACGACATCCACGAGGACAACCTGCTGGAGTGCCCC
CCCGAGGAGCTGGCCAGAGACAGC
SEQ ID NO: 6
HLTF; ΔN Protein
CAICLDSLTVPVITHCAHVFCKPCICQVIQNEQPHAKCPLCRNDIHEDNLLECPPEEL
ARDS
SEQ ID NO: 7
HLTF; ΔNΔC (RING domain only) Coding Sequence
TGCGCCATCTGCCTGGACAGCCTGACCGTGCCCGTGATCACCCACTGCGCCCAC
GTGTTCTGCAAGCCCTGCATCTGCCAGGTGATCCAGAACGAGCAGCCCCACGCC
AAGTGCCCCCTGTGCAGA
SEQ ID NO: 8
HLTF;ANAC (RING domain only) Protein
CAICLDSLTVPVITHCAHVFCKPCICQVIQNEQPHAKCPLCR
I3A: E2-binding domain dead mutation
F20A: Conserved residue F > A point mutation
C38A and C41A: Ligase domain dead mutation
SEQ ID NO: 9
HLTF; ΔE2B (I21A E2-binding mutant) Coding Sequence
AAGAAGCTGATCAGAAAGATGAAGCTGATCCTGAGCAGCGGCAGCGACGAGGA
GTGCGCCGCTTGCCTGGACAGCCTGACCGTGCCCGTGATCACCCACTGCGCCCA
CGTGTTCTGCAAGCCCTGCATCTGCCAGGTGATCCAGAACGAGCAGCCCCACGC
CAAGTGCCCCCTGTGCAGAAACGACATCCACGAGGACAACCTGCTGGAGTGCCC
CCCCGAGGAGCTGGCCAGAGACAGC
SEQ ID NO: 10
HLTE; ΔE2B (I21A E2-binding mutant) Protein
KKLIRKMKLILSSGSDEECAACLDSLTVPVITHCAHVFCKPCICQVIQNEQPHAKCPL
CRNDIHEDNLLECPPEELARDS
SEQ ID NO: 11
HLTF; F38A Coding Sequence
AAGAAGCTGATCAGAAAGATGAAGCTGATCCTGAGCAGCGGCAGCGACGAGGA
GTGCGCCATCTGCCTGGACAGCCTGACCGTGCCCGTGATCACCCACTGCGCCCA
CGTGGCTTGCAAGCCCTGCATCTGCCAGGTGATCCAGAACGAGCAGCCCCACGC
CAAGTGCCCCCTGTGCAGAAACGACATCCACGAGGACAACCTGCTGGAGTGCCC
CCCCGAGGAGCTGGCCAGAGACAGC
SEQ ID NO: 12
HLTF; F38A Protein
KKLIRKMKLILSSGSDEECAICLDSLTVPVITHCAHVACKPCICQVIQNEQPHAKCPL
CRNDIHEDNLLECPPEELARDS
SEQ ID NO: 13
HLTF; ΔZB (C56A and C59A zinc-binding mutant) Coding Sequence
AAGAAGCTGATCAGAAAGATGAAGCTGATCCTGAGCAGCGGCAGCGACGAGGA
GTGCGCCATCTGCCTGGACAGCCTGACCGTGCCCGTGATCACCCACTGCGCCCA
CGTGTTCTGCAAGCCCTGCATCTGCCAGGTGATCCAGAACGAGCAGCCCCACGC
CAAGGCTCCCCTGGCTAGAAACGACATCCACGAGGACAACCTGCTGGAGTGCCC
CCCCGAGGAGCTGGCCAGAGACAGC
SEQ ID NO: 14
HLTF; ΔZB (C56A and C59A zinc-binding mutant) Protein
KKLIRKMKLILSSGSDEECAICLDSLTVPVITHCAHVFCKPCICQVIQNEQPHAKAPL
ARNDIHEDNLLECPPEELARDS
SEQ ID NO: 15
RO52; zf-C3HC4 Coding Sequence
GCCAGCGCCGCCAGACTGACCATGATGTGGGAGGAGGTGACCTGCCCCATCTGC
CTGGACCCCTTCGTGGAGCCCGTGAGCATCGAGTGCGGCCACAGCTTCTGCCAG
GAGTGCATCAGCCAGGTGGGCAAGGGCGGCGGCAGCGTGTGCCCCGTGTGCAG
ACAGAGATTCCTGCTGAAGAACCTGAGACCCAACAGACAGCTGGCCAACATGGT
GAACAACCTGAAGGAGATCAGCCAG
SEQ ID NO: 16
RO52; zf-C3HC4 Protein
ASAARLTMMWEEVTCPICLDPFVEPVSIECGHSFCQECISQVGKGGGSVCPVCRQR
FLLKNLRPNRQLANMVNNLKEISQ
Bold: N- and C-termini
SEQ ID NO: 17
RO52; zf-C3HC4 ΔC Coding Sequence
GCCAGCGCCGCCAGACTGACCATGATGTGGGAGGAGGTGACCTGCCCCATCTGC
CTGGACCCCTTCGTGGAGCCCGTGAGCATCGAGTGCGGCCACAGCTTCTGCCAG
GAGTGCATCAGCCAGGTGGGCAAGGGCGGCGGCAGCGTGTGCCCCGTGTGCAG
ACAGAGA
SEQ ID NO: 18
RO52; zf-C3HC4 ΔC Protein
ASAARLTMMWEEVTCPICLDPFVEPVSIECGHSFCQECISQVGKGGGSVCPVCRQR
SEQ ID NO: 19
RO52; zf-C3HC4 ΔN Coding Sequence
TGCCCCATCTGCCTGGACCCCTTCGTGGAGCCCGTGAGCATCGAGTGCGGCCAC
AGCTTCTGCCAGGAGTGCATCAGCCAGGTGGGCAAGGGCGGCGGCAGCGTGTG
CCCCGTGTGCAGACAGAGATTCCTGCTGAAGAACCTGAGACCCAACAGACAGCT
GGCCAACATGGTGAACAACCTGAAGGAGATCAGCCAG
SEQ ID NO: 20
RO52; zf-C3HC4 ΔN Protein
CPICLDPFVEPVSIECGHSFCQECISQVGKGGGSVCPVCRQRFLLKNLRPNRQLANM
VNNLKEISQ
SEQ ID NO: 21
RO52; zf-C3HC4 ΔNΔC (RING domain only) Coding Sequence
TGCCCCATCTGCCTGGACCCCTTCGTGGAGCCCGTGAGCATCGAGTGCGGCCAC
AGCTTCTGCCAGGAGTGCATCAGCCAGGTGGGCAAGGGCGGCGGCAGCGTGTG
CCCCGTGTGCAGACAGAGA
SEQ ID NO: 22
RO52; zf-C3HC4 ΔNΔC (RING domain only) Protein
CPICLDPFVEPVSIECGHSFCQECISQVGKGGGSVCPVCRQR
SEQ ID NO: 23
HLTF Full Protein
MSWMFKRDPVWKYLQTVQYGVHGNFPRLSYPTFFPRFEFQDVIPPDDFLTSDEEVD
SVLFGSLRGHVVGLRYYTGVVNNNEMVALQRDPNNPYDKNAIKVNNVNGNQVGH
LKKELAGALAYIMDNKLAQIEGVVPFGANNAFTMPLHMTFWGKEENRKAVSDQLK
KHGFKLGPAPKTLGFNLESGWGSGRAGPSYSMPVHAAVQMTTEQLKTEFDKLFED
LKEDDKTHEMEPAEAIETPLLPHQKQALAWMVSRENSKELPPFWEQRNDLYYNTIT
NFSEKDRPENVHGGILADDMGLGKTLTAIAVILTNFHDGRPLPIERVKKNLLKKEYN
VNDDSMKLGGNNTSEKADGLSKDASRCSEQPSISDIKEKSKFRMSELSSSRPKRRKT
AVQYIESSDSEEIETSELPQKMKGKLKNVQSETKGRAKAGSSKVIEDVAFACALTSS
VPTTKKKMLKKGACAVEGSKKTDVEERPRTTLIICPLSVLSNWIDQFGQHIKSDVHL
NFYVYYGPDRIREPALLSKQDIVLTTYNILTHDYGTKGDSPLHSIRWLRVILDEGHAI
RNPNAQQTKAVLDLESERRWVLTGTPIQNSLKDLWSLLSFLKLKPFIDREWWHRTI
QRPVTMGDEGGLRRLQSLIKNITLRRTKTSKIKGKPVLELPERKVFIQHITLSDEERKI
YQSVKNEGRATIGRYFNEGTVLAHYADVLGLLLRLRQICCHTYLLTNAVSSNGPSG
NDTPEELRKKLIRKMKLILSSGSDEECAICLDSLTVPVITHCAHVFCKPCICQVIQN
EQPHAKCPLCRNDIHEDNLLECPPEELARDSEKKSDMEWTSSSKINALMHALTDLR
KKNPNIKSLVVSQFTTFLSLIEIPLKASGFVFTRLDGSMAQKKRVESIQCFQNTEAGSP
TIMLLSLKAGGVGLNLSAASRVFLMDPAWNPAAEDQCFDRCHRLGQKQEVIITKFI
VKDSVEENMLKIQNKKRELAAGAFGTKKPNADEMKQAKINEIRTLIDL
Lacking in exemplary HLTF; zf-C3HC4 peptides:
Positions 1-741
Positions 822-1009
SEQ ID NO: 24
RO52 Full Protein
MASAARLTMMWEEVTCPICLDPFVEPVSIECGHSFCQECISQVGKGGGSVCPVC
RQRFLLKNLRPNRQLANMVNNLKEISQEAREGTQGERCAVHGERLHLFCEKDGKA
LCWVCAQSRKHRDHAMVPLEEAAQEYQEKLQVALGELRRKQELAEKLEVEIAIKR
ADWKKTVETQKSRIHAEFVQQKNFLVEEEQRQLQELEKDEREQLRILGEKEAKLAQ
QSQALQELISELDRRCHSSALELLQEVIIVLERSESWNLKDLDITSPELRSVCHVPGLK
KMLRTCAVHITLDPDTANPWLILSEDRRQVRLGDTQQSIPGNEERFDSYPMVLGAQ
HFHSGKHYWEVDVTGKEAWDLGVCRDSVRRKGHFLLSSKSGFWTIWLWNKQKYE
AGTYPQTPLHLQVPPCQVGIFLDYEAGMVSFYNITDHGSLIYSFSECAFTGPLRPFFSP
GFNDGGKNTAPLTLCPLNIGSQGSTDY
Lacking in exemplary RO52; zf-C3HC4 peptides:
Position 1
Positions 82-475
SEQ ID NO: 25
Ef1a-Cas9-ABI-T2A-GFP-P2A-Blast
GTGCCCGTCAGTGGGCAGAGCGCACATCGCCCACAGTCCCCGAGAAGTTGGGGG
GAGGGGTCGGCAATTGAACCGGTGCCTAGAGAAGGTGGCGCGGGGTAAACTGG
GAAAGTGATGTCGTGTACTGGCTCCGCCTTTTTCCCGAGGGTGGGGGAGAACCG
TATATAAGTGCAGTAGTCGCCGTGAACGTTCTTTTTCGCAACGGGTTTGCCGCCA
GAACACAGGTAAGTGCCGTGTGTGGTTCCCGCGGGCCTGGCCTCTTTACGGGTT
ATGGCCCTTGCGTGCCTTGAATTACTTCCACCTGGCTGCAGTACGTGATTCTTGA
TCCCGAGCTTCGGGTTGGAAGTGGGTGGGAGAGTTCGAGGCCTTGCGCTTAAGG
AGCCCCTTCGCCTCGTGCTTGAGTTGAGGCCTGGCCTGGGCGCTGGGGCCGCCG
CGTGCGAATCTGGTGGCACCTTCGCGCCTGTCTCGCTGCTTTCGATAAGTCTCTA
GCCATTTAAAATTTTTGATGACCTGCTGCGACGCTTTTTTTCTGGCAAGATAGTC
TTGTAAATGCGGGCCAAGATCTGCACACTGGTATTTCGGTTTTTGGGGCCGCGG
GCGGCGACGGGGCCCGTGCGTCCCAGCGCACATGTTCGGCGAGGCGGGGCCTGC
GAGCGCGGCCACCGAGAATCGGACGGGGGTAGTCTCAAGCTGGCCGGCCTGCTC
TGGTGCCTGGCCTCGCGCCGCCGTGTATCGCCCCGCCCTGGGCGGCAAGGCTGG
CCCGGTCGGCACCAGTTGCGTGAGCGGAAAGATGGCCGCTTCCCGGCCCTGCTG
CAGGGAGCTCAAAATGGAGGACGCGGCGCTCGGGAGAGCGGGCGGGTGAGTCA
CCCACACAAAGGAAAAGGGCCTTTCCGTCCTCAGCCGTCGCTTCATGTGACTCC
ACGGAGTACCGGGCGCCGTCCAGGCACCTCGATTAGTTCTCGcGCTTTTGGAGTA
CGTCGTCTTTAGGTTGGGGGGAGGGGTTTTATGCGATGGAGTTTCCCCACACTGA
GTGGGTGGAGACTGAAGTTAGGCCAGCTTGGCACTTGATGTAATTCTCCTTGGA
ATTTGCCCTTTTTGAGTTTGGATCTTGGTTCATTCTCAAGCCTCAGACAGTGGTTC
AAAGTTTTTTTCTTCCATTTCAGGTGTCGTGAgCTAGAGCGCTGCCACCATGGAC
AAGAAGTACAGCATCGGCCTGGACATCGGCACCAACTCTGTGGGCTGGGCCGTG
ATCACCGACGAGTACAAGGTGCCCAGCAAGAAATTCAAGGTGCTGGGCAACAC
CGACCGGCACAGCATCAAGAAGAACCTGATCGGAGCCCTGCTGTTCGACAGCGG
CGAAACAGCCGAGGCCACCCGGCTGAAGAGAACCGCCAGAAGAAGATACACCA
GACGGAAGAACCGGATCTGCTATCTGCAAGAGATCTTCAGCAACGAGATGGCCA
AGGTGGACGACAGCTTCTTCCACAGACTGGAAGAGTCCTTCCTGGTGGAAGAGG
ATAAGAAGCACGAGCGGCACCCCATCTTCGGCAACATCGTGGACGAGGTGGCCT
ACCACGAGAAGTACCCCACCATCTACCACCTGAGAAAGAAACTGGTGGACAGC
ACCGACAAGGCCGACCTGCGGCTGATCTATCTGGCCCTGGCCCACATGATCAAG
TTCCGGGGCCACTTCCTGATCGAGGGCGACCTGAACCCCGACAACAGCGACGTG
GACAAGCTGTTCATCCAGCTGGTGCAGACCTACAACCAGCTGTTCGAGGAAAAC
CCCATCAACGCCAGCGGCGTGGACGCCAAGGCCATCCTGTCTGCCAGACTGAGC
AAGAGCAGACGGCTGGAAAATCTGATCGCCCAGCTGCCCGGCGAGAAGAAGAA
TGGCCTGTTCGGAAACCTGATTGCCCTGAGCCTGGGCCTGACCCCCAACTTCAA
GAGCAACTTCGACCTGGCCGAGGATGCCAAACTGCAGCTGAGCAAGGACACCT
ACGACGACGACCTGGACAACCTGCTGGCCCAGATCGGCGACCAGTACGCCGACC
TGTTTCTGGCCGCCAAGAACCTGTCCGACGCCATCCTGCTGAGCGACATCCTGA
GAGTGAACACCGAGATCACCAAGGCCCCCCTGAGCGCCTCTATGATCAAGAGAT
ACGACGAGCACCACCAGGACCTGACCCTGCTGAAAGCTCTCGTGCGGCAGCAGC
TGCCTGAGAAGTACAAAGAGATTTTCTTCGACCAGAGCAAGAACGGCTACGCCG
GCTACATTGACGGCGGAGCCAGCCAGGAAGAGTTCTACAAGTTCATCAAGCCCA
TCCTGGAAAAGATGGACGGCACCGAGGAACTGCTCGTGAAGCTGAACAGAGAG
GACCTGCTGCGGAAGCAGCGGACCTTCGACAACGGCAGCATCCCCCACCAGATC
CACCTGGGAGAGCTGCACGCCATTCTGCGGCGGCAGGAAGATTTTTACCCATTC
CTGAAGGACAACCGGGAAAAGATCGAGAAGATCCTGACCTTCCGCATCCCCTAC
TACGTGGGCCCTCTGGCCAGGGGAAACAGCAGATTCGCCTGGATGACCAGAAA
GAGCGAGGAAACCATCACCCCCTGGAACTTCGAGGAAGTGGTGGACAAGGGCG
CTTCCGCCCAGAGCTTCATCGAGCGGATGACCAACTTCGATAAGAACCTGCCCA
ACGAGAAGGTGCTGCCCAAGCACAGCCTGCTGTACGAGTACTTCACCGTGTATA
ACGAGCTGACCAAAGTGAAATACGTGACCGAGGGAATGAGAAAGCCCGCCTTC
CTGAGCGGCGAGCAGAAAAAGGCCATCGTGGACCTGCTGTTCAAGACCAACCG
GAAAGTGACCGTGAAGCAGCTGAAAGAGGACTACTTCAAGAAAATCGAGTGCT
TCGACTCCGTGGAAATCTCCGGCGTGGAAGATCGGTTCAACGCCTCCCTGGGCA
CATACCACGATCTGCTGAAAATTATCAAGGACAAGGACTTCCTGGACAATGAGG
AAAACGAGGACATTCTGGAAGATATCGTGCTGACCCTGACACTGTTTGAGGACA
GAGAGATGATCGAGGAACGGCTGAAAACCTATGCCCACCTGTTCGACGACAAA
GTGATGAAGCAGCTGAAGCGGCGGAGATACACCGGCTGGGGCAGGCTGAGCCG
GAAGCTGATCAACGGCATCCGGGACAAGCAGTCCGGCAAGACAATCCTGGATTT
CCTGAAGTCCGACGGCTTCGCCAACAGAAACTTCATGCAGCTGATCCACGACGA
CAGCCTGACCTTTAAAGAGGACATCCAGAAAGCCCAGGTGTCCGGCCAGGGCG
ATAGCCTGCACGAGCACATTGCCAATCTGGCCGGCAGCCCCGCCATTAAGAAGG
GCATCCTGCAGACAGTGAAGGTGGTGGACGAGCTCGTGAAAGTGATGGGCCGG
CACAAGCCCGAGAACATCGTGATCGAAATGGCCAGAGAGAACCAGACCACCCA
GAAGGGACAGAAGAACAGCCGCGAGAGAATGAAGCGGATCGAAGAGGGCATC
AAAGAGCTGGGCAGCCAGATCCTGAAAGAACACCCCGTGGAAAACACCCAGCT
GCAGAACGAGAAGCTGTACCTGTACTACCTGCAGAATGGGCGGGATATGTACGT
GGACCAGGAACTGGACATCAACCGGCTGTCCGACTACGATGTGGACCATATCGT
GCCTCAGAGCTTTCTGAAGGACGACTCCATCGACAACAAGGTGCTGACCAGAAG
CGACAAGAACCGGGGCAAGAGCGACAACGTGCCCTCCGAAGAGGTCGTGAAGA
AGATGAAGAACTACTGGCGGCAGCTGCTGAACGCCAAGCTGATTACCCAGAGA
AAGTTCGACAATCTGACCAAGGCCGAGAGAGGCGGCCTGAGCGAACTGGATAA
GGCCGGCTTCATCAAGAGACAGCTGGTGGAAACCCGGCAGATCACAAAGCACG
TGGCACAGATCCTGGACTCCCGGATGAACACTAAGTACGACGAGAATGACAAG
CTGATCCGGGAAGTGAAAGTGATCACCCTGAAGTCCAAGCTGGTGTCCGATTTC
CGGAAGGATTTCCAGTTTTACAAAGTGCGCGAGATCAACAACTACCACCACGCC
CACGACGCCTACCTGAACGCCGTCGTGGGAACCGCCCTGATCAAAAAGTACCCT
AAGCTGGAAAGCGAGTTCGTGTACGGCGACTACAAGGTGTACGACGTGCGGAA
GATGATCGCCAAGAGCGAGCAGGAAATCGGCAAGGCTACCGCCAAGTACTTCTT
CTACAGCAACATCATGAACTTTTTCAAGACCGAGATTACCCTGGCCAACGGCGA
GATCCGGAAGCGGCCTCTGATCGAGACAAACGGCGAAACCGGGGAGATCGTGT
GGGATAAGGGCCGGGATTTTGCCACCGTGCGGAAAGTGCTGAGCATGCCCCAAG
TGAATATCGTGAAAAAGACCGAGGTGCAGACAGGCGGCTTCAGCAAAGAGTCT
ATCCTGCCCAAGAGGAACAGCGATAAGCTGATCGCCAGAAAGAAGGACTGGGA
CCCTAAGAAGTACGGCGGCTTCGACAGCCCCACCGTGGCCTATTCTGTGCTGGT
GGTGGCCAAAGTGGAAAAGGGCAAGTCCAAGAAACTGAAGAGTGTGAAAGAGC
TGCTGGGGATCACCATCATGGAAAGAAGCAGCTTCGAGAAGAATCCCATCGACT
TTCTGGAAGCCAAGGGCTACAAAGAAGTGAAAAAGGACCTGATCATCAAGCTG
CCTAAGTACTCCCTGTTCGAGCTGGAAAACGGCCGGAAGAGAATGCTGGCCTCT
GCCGGCGAACTGCAGAAGGGAAACGAACTGGCCCTGCCCTCCAAATATGTGAA
CTTCCTGTACCTGGCCAGCCACTATGAGAAGCTGAAGGGCTCCCCCGAGGATAA
TGAGCAGAAACAGCTGTTTGTGGAACAGCACAAGCACTACCTGGACGAGATCAT
CGAGCAGATCAGCGAGTTCTCCAAGAGAGTGATCCTGGCCGACGCTAATCTGGA
CAAAGTGCTGTCCGCCTACAACAAGCACCGGGATAAGCCCATCAGAGAGCAGG
CCGAGAATATCATCCACCTGTTTACCCTGACCAATCTGGGAGCCCCTGCCGCCTT
CAAGTACTTTGACACCACCATCGACCGGAAGAGGTACACCAGCACCAAAGAGG
TGCTGGACGCCACCCTGATCCACCAGAGCATCACCGGCCTGTACGAGACACGGA
TCGACCTGTCTCAGCTGGGAGGCGACTCTAGAGGTGGAGGCTCTGGAACGCGTG
TGCCTTTGTATGGTTTTACTTCGATTTGTGGAAGAAGACCTGAGATGGAAGcTGC
TGTTTCGACTATACCAAGATTCCTTCAATCTTCCTCTGGTTCGATGTTAGATGGTC
GGTTTGATCCTCAATCCGCCGCTCATTTCTTCGGTGTTTACGACGGCCATGGCGG
TTCTCAGGTAGCGAACTATTGTAGAGAGAGGATGCATTTGGCTTTGGCGGAGGA
GATAGCTAAGGAGAAACCGATGCTCTGCGATGGTGATACGTGGCTGGAGAAGT
GGAAGAAAGCTCTTTTCAACTCGTTCCTGAGAGTTGACTCGGAGATTGAGTCAG
TTGCGCCGGAAACGGTTGGGTCAACGTCGGTGGTTGCCGTTGTTTTCCCGTCGCA
CATCTTCGTCGCTAACTGCGGTGACTCCAGAGCCGTTCTTTGCCGCGGCAAAACT
GCACTTCCATTATCCGTTGACCATAAACCGGATAGAGAAGATGAAGCTGCGAGG
ATTGAAGCCGCAGGAGGGAAAGTGATTCAGTGGAATGGAGCTCGTGTTTTCGGT
GTTCTCGCCATGTCGAGATCCATTGGCGATAGATACTTGAAACCATCCATCATTC
CTGATCCGGAAGTGACGGCTGTGAAGAGAGTAAAAGAAGATGATTGTCTGATTT
TGGCGAGTGACGGGGTTTGGGATGTAATGACGGATGAAGAAGCGTGTGAGATG
GCAAGGAAGCGGATTCTCTTGTGGCACAAGAAAAACGCGGTGGCTGGGGATGC
ATCGTTGCTCGCGGATGAGCGGAGAAAGGAAGGGAAAGATCCTGCGGCGATGT
CCGCGGCTGAGTATTTGTCAAAGCTGGCGATACAGAGAGGAAGCAAAGACAAC
ATAAGTGTGGTGGTGGTTGATTTGAAGgttAAGCGACCTGCCGCCACAAAGAAGG
CTGGACAGGCTAAGAAGAAGAAAGATTACAAAGACGATGACGATAAGggatccGA
GGGCAGAGGAAGTCTGCTAACATGCGGTGACGTCGAGGAGAATCCTGGCCCAcc
ggtcgccaccatggtgagcaagggcgaggagctgttcaccggggtggtgcccatcctggtcgagctggacggcgacgtaaacgg
ccacaagttcagcgtgtcTggcgagggcgagggcgatgccacctacggcaagctgaccctgaagttcatctgcaccaccggcaa
gctgcccgtgccctggcccaccctcgtgaccaccctgacctacggcgtgcagtgcttcagccgctaccccgaccacatgaagcagc
acgacttcttcaagtccgccatgcccgaaggctacgtccaggagcgcaccatcttcttcaaggacgacggcaactacaagacccgc
gccgaggtgaagttcgagggcgacaccctggtgaaccgcatcgagctgaagggcatcgacttcaaggaggacggcaacatcctg
gggcacaagctggagtacaactacaacagccacaacgtctatatcatggccgacaagcagaagaacggcatcaaggcgaacttca
agatccgccacaacatcgaggacggcagcgtgcagctcgccgaccactaccagcagaacacccccatcggcgacggccccgtg
ctgctgcccgacaaccactacctgagcacccagtccgccctgagcaaagaccccaacgagaagcgcgatcacatggtcctgctgg
agttcgtgaccgccgccgggatcactctcggcatggacgagctCtacaagGGAAGCGGAGCTACTAACTTCA
GCCTGCTGAAGCAGGCTGGTGACGTGGAGGAGAACCCTGGACCTatggccaagcctttgt
ctcaagaagaatccaccctcattgaaagagcaacggctacaatcaacagcatccccatctctgaagactacagcgtcgccagcgca
gctctctctagcgacggccgcatcttcactggtgtcaatgtatatcattttactgggggaccttgtgcagaactcgtggtgctgggcact
gctgctgctgcggcagctggcaacctgacttgtatcgtcgcgatcggaaatgagaacaggggcatcttgagcccctgcggacggtg
ccgacaggtgcttctcgatctgcatcctgggatcaaagccatagtgaaggacagtgatggacagccgacggcagttgggattcgtga
attgctgccctctggttatgtgtgggagggctaa
EF1a Promoter: 1-1173
Cas9 Coding Sequence: 1190-5293
ABI Coding Sequence: 5315-6214
Nuclear Localization Signal: 6218-6265
Flag Tag: 6266-6289
T2A Self-Cleaving Peptide Sequence: 6296-6349
EGFP Coding Sequence: 6362-7078
P2A Self-Cleaving Peptide Sequence: 7088-7144
Blasticidin Resistance Coding Sequence: 7145-7543
SEQ ID NO: 26
Ef1a-tagBFP-ABI-T2A-GFP-P2A-Blast
GTGCCCGTCAGTGGGCAGAGCGCACATCGCCCACAGTCCCCGAGAAGTTGGGGG
GAGGGGTCGGCAATTGAACCGGTGCCTAGAGAAGGTGGCGCGGGGTAAACTGG
GAAAGTGATGTCGTGTACTGGCTCCGCCTTTTTCCCGAGGGTGGGGGAGAACCG
TATATAAGTGCAGTAGTCGCCGTGAACGTTCTTTTTCGCAACGGGTTTGCCGCCA
GAACACAGGTAAGTGCCGTGTGTGGTTCCCGCGGGCCTGGCCTCTTTACGGGTT
ATGGCCCTTGCGTGCCTTGAATTACTTCCACCTGGCTGCAGTACGTGATTCTTGA
TCCCGAGCTTCGGGTTGGAAGTGGGTGGGAGAGTTCGAGGCCTTGCGCTTAAGG
AGCCCCTTCGCCTCGTGCTTGAGTTGAGGCCTGGCCTGGGCGCTGGGGCCGCCG
CGTGCGAATCTGGTGGCACCTTCGCGCCTGTCTCGCTGCTTTCGATAAGTCTCTA
GCCATTTAAAATTTTTGATGACCTGCTGCGACGCTTTTTTTCTGGCAAGATAGTC
TTGTAAATGCGGGCCAAGATCTGCACACTGGTATTTCGGTTTTTGGGGCCGCGG
GCGGCGACGGGGCCCGTGCGTCCCAGCGCACATGTTCGGCGAGGCGGGGCCTGC
GAGCGCGGCCACCGAGAATCGGACGGGGGTAGTCTCAAGCTGGCCGGCCTGCTC
TGGTGCCTGGCCTCGCGCCGCCGTGTATCGCCCCGCCCTGGGCGGCAAGGCTGG
CCCGGTCGGCACCAGTTGCGTGAGCGGAAAGATGGCCGCTTCCCGGCCCTGCTG
CAGGGAGCTCAAAATGGAGGACGCGGCGCTCGGGAGAGCGGGCGGGTGAGTCA
CCCACACAAAGGAAAAGGGCCTTTCCGTCCTCAGCCGTCGCTTCATGTGACTCC
ACGGAGTACCGGGCGCCGTCCAGGCACCTCGATTAGTTCTCGcGCTTTTGGAGTA
CGTCGTCTTTAGGTTGGGGGGAGGGGTTTTATGCGATGGAGTTTCCCCACACTGA
GTGGGTGGAGACTGAAGTTAGGCCAGCTTGGCACTTGATGTAATTCTCCTTGGA
ATTTGCCCTTTTTGAGTTTGGATCTTGGTTCATTCTCAAGCCTCAGACAGTGGTTC
AAAGTTTTTTTCTTCCATTTCAGGTGTCGTGAgCTAGAGCGCTGCCACCATGAGC
GAGCTGATTAAGGAGAACATGCACATGAAGCTGTACATGGAGGGCACCGTGGA
CAACCATCACTTCAAGTGCACATCCGAGGGCGAAGGCAAGCCCTACGAGGGCA
CCCAGACCATGAGAATCAAGGTGGTCGAGGGCGGCCCTCTCCCCTTCGCCTTCG
ACATCCTGGCTACTAGCTTCCTCTACGGCAGCAAGACCTTCATCAACCACACCCA
GGGCATCCCCGACTTCTTCAAGCAGTCCTTCCCTGAGGGCTTCACATGGGAGAG
AGTCACCACATACGAAGACGGGGGCGTGCTGACCGCTACCCAGGACACCAGCCT
CCAGGACGGCTGCCTCATCTACAACGTCAAGATCAGAGGGGTGAACTTCACATC
CAACGGCCCTGTGATGCAGAAGAAAACACTCGGCTGGGAGGCCTTCACCGAaAC
GCTGTACCCCGCTGACGGCGGCCTGGAAGGCAGAAACGACATGGCCCTGAAGCT
CGTGGGCGGGAGCCATCTGATCGCAAACATCAAGACCACATATAGATCCAAGA
AACCCGCTAAGAACCTCAAGATGCCTGGCGTCTACTATGTGGACTACAGACTGG
AAAGAATCAAGGAGGCCAACAACGAGACCTACGTCGAGCAGCACGAGGTGGCA
GTGGCCAGATACTGCGACCTCCCTAGCAAACTGGGGCACAAGCTTAATTCTAGA
GGTGGAGGCTCTGGAACGCGTGTGCCTTTGTATGGTTTTACTTCGATTTGTGGAA
GAAGACCTGAGATGGAAGCTGCTGTTTCGACTATACCAAGATTCCTTCAATCTTC
CTCTGGTTCGATGTTAGATGGTCGGTTTGATCCTCAATCCGCCGCTCATTTCTTCG
GTGTTTACGACGGCCATGGCGGTTCTCAGGTAGCGAACTATTGTAGAGAGAGGA
TGCATTTGGCTTTGGCGGAGGAGATAGCTAAGGAGAAACCGATGCTCTGCGATG
GTGATACGTGGCTGGAGAAGTGGAAGAAAGCTCTTTTCAACTCGTTCCTGAGAG
TTGACTCGGAGATTGAGTCAGTTGCGCCGGAAACGGTTGGGTCAACGTCGGTGG
TTGCCGTTGTTTTCCCGTCGCACATCTTCGTCGCTAACTGCGGTGACTCCAGAGC
CGTTCTTTGCCGCGGCAAAACTGCACTTCCATTATCCGTTGACCATAAACCGGAT
AGAGAAGATGAAGCTGCGAGGATTGAAGCCGCAGGAGGGAAAGTGATTCAGTG
GAATGGAGCTCGTGTTTTCGGTGTTCTCGCCATGTCGAGATCCATTGGCGATAGA
TACTTGAAACCATCCATCATTCCTGATCCGGAAGTGACGGCTGTGAAGAGAGTA
AAAGAAGATGATTGTCTGATTTTGGCGAGTGACGGGGTTTGGGATGTAATGACG
GATGAAGAAGCGTGTGAGATGGCAAGGAAGCGGATTCTCTTGTGGCACAAGAA
AAACGCGGTGGCTGGGGATGCATCGTTGCTCGCGGATGAGCGGAGAAAGGAAG
GGAAAGATCCTGCGGCGATGTCCGCGGCTGAGTATTTGTCAAAGCTGGCGATAC
AGAGAGGAAGCAAAGACAACATAAGTGTGGTGGTGGTTGATTTGAAGgttAAGCG
ACCTGCCGCCACAAAGAAGGCTGGACAGGCTAAGAAGAAGAAAGATTACAAAG
ACGATGACGATAAGggatccGAGGGCAGAGGAAGTCTGCTAACATGCGGTGACGT
CGAGGAGAATCCTGGCCCAccggtcgccaccatggtgagcaagggcgaggagctgttcaccggggtggtgccc
atcctggtcgagctggacggcgacgtaaacggccacaagttcagcgtgtcTggcgagggcgagggcgatgccacctacggcaa
gctgaccctgaagttcatctgcaccaccggcaagctgcccgtgccctggcccaccctcgtgaccaccctgacctacggcgtgcagt
gcttcagccgctaccccgaccacatgaagcagcacgacttcttcaagtccgccatgcccgaaggctacgtccaggagcgcaccatc
ttcttcaaggacgacggcaactacaagacccgcgccgaggtgaagttcgagggcgacaccctggtgaaccgcatcgagctgaagg
gcatcgacttcaaggaggacggcaacatcctggggcacaagctggagtacaactacaacagccacaacgtctatatcatggccgac
aagcagaagaacggcatcaaggcgaacttcaagatccgccacaacatcgaggacggcagcgtgcagctcgccgaccactaccag
cagaacacccccatcggcgacggccccgtgctgctgcccgacaaccactacctgagcacccagtccgccctgagcaaagacccc
aacgagaagcgcgatcacatggtcctgctggagttcgtgaccgccgccgggatcactctcggcatggacgagctCtacaagGG
AAGCGGAGCTACTAACTTCAGCCTGCTGAAGCAGGCTGGTGACGTGGAGGAGA
ACCCTGGACCTatggccaagcctttgtctcaagaagaatccaccctcattgaaagagcaacggctacaatcaacagcatcc
ccatctctgaagactacagcgtcgccagcgcagctctctctagcgacggccgcatcttcactggtgtcaatgtatatcattttactgggg
gaccttgtgcagaactcgtggtgctgggcactgctgctgctgcggcagctggcaacctgacttgtatcgtcgcgatcggaaatgaga
acaggggcatcttgagcccctgcggacggtgccgacaggtgcttctcgatctgcatcctgggatcaaagccatagtgaaggacagt
gatggacagccgacggcagttgggattcgtgaattgctgccctctggttatgtgtgggagggctaa
EF1a Promoter: 1-1173
tagBFP Coding Sequence: 1190-1888
ABI Coding Sequence: 1910-2809
Nuclear Localization Signal: 2813-2860
Flag Tag: 2861-2884
T2A Self-Cleaving Peptide Sequence: 2891-2944
EGFP Coding Sequence: 2957-3673
P2A Self-Cleaving Peptide Sequence: 3683-3739
Blasticidin Resistance Coding Sequence: 3740-4138
SEQ ID NO: 27
Ef1a-HA-PYL1-NLS-HLTF/RO52; zfC3HC4-T2A-mCherry-P2A-Puro
GTGCCCGTCAGTGGGCAGAGCGCACATCGCCCACAGTCCCCGAGAAGTTGGGGG
GAGGGGTCGGCAATTGAACCGGTGCCTAGAGAAGGTGGCGCGGGGTAAACTGG
GAAAGTGATGTCGTGTACTGGCTCCGCCTTTTTCCCGAGGGTGGGGGAGAACCG
TATATAAGTGCAGTAGTCGCCGTGAACGTTCTTTTTCGCAACGGGTTTGCCGCCA
GAACACAGGTAAGTGCCGTGTGTGGTTCCCGCGGGCCTGGCCTCTTTACGGGTT
ATGGCCCTTGCGTGCCTTGAATTACTTCCACCTGGCTGCAGTACGTGATTCTTGA
TCCCGAGCTTCGGGTTGGAAGTGGGTGGGAGAGTTCGAGGCCTTGCGCTTAAGG
AGCCCCTTCGCCTCGTGCTTGAGTTGAGGCCTGGCCTGGGCGCTGGGGCCGCCG
CGTGCGAATCTGGTGGCACCTTCGCGCCTGTCTCGCTGCTTTCGATAAGTCTCTA
GCCATTTAAAATTTTTGATGACCTGCTGCGACGCTTTTTTTCTGGCAAGATAGTC
TTGTAAATGCGGGCCAAGATCTGCACACTGGTATTTCGGTTTTTGGGGCCGCGG
GCGGCGACGGGGCCCGTGCGTCCCAGCGCACATGTTCGGCGAGGCGGGGCCTGC
GAGCGCGGCCACCGAGAATCGGACGGGGGTAGTCTCAAGCTGGCCGGCCTGCTC
TGGTGCCTGGCCTCGCGCCGCCGTGTATCGCCCCGCCCTGGGCGGCAAGGCTGG
CCCGGTCGGCACCAGTTGCGTGAGCGGAAAGATGGCCGCTTCCCGGCCCTGCTG
CAGGGAGCTCAAAATGGAGGACGCGGCGCTCGGGAGAGCGGGCGGGTGAGTCA
CCCACACAAAGGAAAAGGGCCTTTCCGTCCTCAGCCGTCGCTTCATGTGACTCC
ACGGAGTACCGGGCGCCGTCCAGGCACCTCGATTAGTTCTCGCGCTTTTGGAGT
ACGTCGTCTTTAGGTTGGGGGGAGGGGTTTTATGCGATGGAGTTTCCCCACACTG
AGTGGGTGGAGACTGAAGTTAGGCCAGCTTGGCACTTGATGTAATTCTCCTTGG
AATTTGCCCTTTTTGAGTTTGGATCTTGGTTCATTCTCAAGCCTCAGACAGTGGTT
CAAAGTTTTTTTCTTCCATTTCAGGTGTCGTGAGCTAGCCACCATGTATCCCTAT
GACGTGCCCGATTATGCCGGTGGGGGCGCGCCAACTCAAGACGAATTCACCCAA
CTCTCCCAATCAATCGCCGAGTTCCACACGTACCAACTCGGTAACGGCCGTTGCT
CATCTCTCCTAGCTCAGCGAATCCACGCGCCGCCGGAAACAGTATGGTCCGTGG
TGAGTCGTTTCGATAGGCCACAGATTTACAAACACTTCATCAAAAGCTGTAACG
TGAGTGAAGATTTCGAGATGCGAGTGGGATGCACGCGCGACGTGAACGTGATA
AGTGGATTACCGGCGAATACCTCTCGAGAGAGATTAGATCTGTTGGACGATGAT
CGGAGAGTGACTGGGTTTAGTATAACCGGTGGTGAACATAGGCTGAGGAATTAT
AAATCGGTTACGACGGTTCATAGATTTGAGAAAGAAGAAGAAGAAGAAAGGAT
CTGGACCGTTGTTTTGGAATCTTATGTTGTTGATGTACCGGAAGGTAATTCGGAG
GAAGATACGAGATTGTTTGCTGATACGGTTATTAGATTGAATCTTCAGAAACTTG
CTTCGATCACTGAAGCTATGAACAGCGGAGGAGGAGGTAGCGGACCTAAGAAA
AAGAGGAAGGTGGCGGCCGCTGGATCCGGATCAGGCTCCNNNNNNNNNNNNNN
NNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNN
NNNNNNNNNNNNNNNNNNNNNNGGATCTGGAGAGGGAAGGGGAAGCCTCCTA
ACTTGCGGAGATGTCGAGGAGAATCCTGGCCCAATGGTGAGCAAGGGCGAGGA
GGATAACATGGCCATCATCAAGGAGTTCATGCGCTTCAAGGTGCACATGGAGGG
CTCCGTGAACGGCCACGAGTTCGAGATCGAGGGCGAGGGCGAGGGCCGCCCCT
ACGAGGGCACCCAGACCGCCAAGCTGAAGGTGACCAAGGGTGGCCCCCTGCCC
TTCGCCTGGGACATCCTGTCCCCTCAGTTCATGTACGGCTCCAAGGCCTACGTGA
AGCACCCCGCCGACATCCCCGACTACTTGAAGCTGTCCTTCCCCGAGGGCTTCA
AGTGGGAGCGCGTGATGAACTTCGAGGACGGCGGCGTGGTGACCGTGACCCAG
GACTCCTCCCTGCAGGACGGCGAGTTCATCTACAAGGTGAAGCTGCGCGGCACC
AACTTCCCCTCCGACGGCCCCGTAATGCAGAAGAAGACCATGGGCTGGGAGGCC
TCCTCCGAGCGGATGTACCCCGAGGACGGCGCCCTGAAGGGCGAGATCAAGCA
GAGGCTGAAGCTGAAGGACGGCGGCCACTACGACGCTGAGGTCAAGACCACCT
ACAAGGCCAAGAAGCCCGTGCAGCTGCCCGGCGCCTACAACGTCAACATCAAGT
TGGACATCACCTCCCACAACGAGGACTACACCATCGTGGAACAGTACGAACGCG
CCGAGGGCCGCCACTCCACCGGCGGCATGGACGAGCTGTACAAGGGAAGCGGA
GCTACTAACTTCAGCCTGCTGAAGCAGGCTGGTGACGTGGAGGAGAACCCTGGA
CCTATGACCGAGTACAAGCCCACGGTGCGCCTCGCCACCCGCGACGACGTCCCC
CGGGCCGTACGCACCCTCGCCGCCGCGTTCGCCGACTACCCCGCCACGCGCCAC
ACCGTCGACCCGGACCGCCACATCGAGCGGGTCACCGAGCTGCAAGAACTCTTC
CTCACGCGCGTCGGGCTCGACATCGGCAAGGTGTGGGTCGCGGACGACGGCGCC
GCGGTGGCGGTCTGGACCACGCCGGAGAGCGTCGAAGCGGGGGCGGTGTTCGC
CGAGATCGGCCCGCGCATGGCCGAGTTGAGCGGTTCCCGGCTGGCCGCGCAGCA
ACAGATGGAAGGCCTCCTGGCGCCGCACCGGCCCAAGGAGCCCGCGTGGTTCCT
GGCCACCGTCGGCGTATCGCCCGACCACCAGGGCAAGGGTCTGGGCAGCGCCGT
CGTGCTCCCCGGAGTGGAGGCGGCCGAGCGCGCCGGGGTGCCCGCCTTCCTGGA
GACCTCCGCGCCCCGCAACCTCCCCTTCTACGAGCGGCTCGGCTTCACCGTCACC
GCCGACGTCGAGGTGCCCGAAGGACCGCGCACCTGGTGCATGACCCGCAAGCCC
GGTGCCTAA
EF1a Promoter: 1-1173
HA Tag: 1186-1212
PYL1 Coding Sequence: 1213-1758
Nuclear Localization Signal: 1780-1800
Placeholder Sequence for SEQ ID NOS: 1, 3, 5, 7, 9, 11, 13, 15, 17, 19, and 21: 1828-
2067 (Not all Ns will be present for truncated peptides such as SEQ ID NOS: 3, 5, 7,
17, 19, and 21)
T2A Self-Cleaving Sequence: 2077-2130
mCherry CodingSequence: 2131-2838
P2A Self-Cleaving Peptide Sequence: 2848-2904
Puromycin Resistance Coding Sequence: 2905-3504
SEQ ID NO: 28
Ef1a-HA-PYL1-HLTF/RO52; zfC3HC4-T2A-mCherry-P2A-Puro (No NLS)
GTGCCCGTCAGTGGGCAGAGCGCACATCGCCCACAGTCCCCGAGAAGTTGGGGG
GAGGGGTCGGCAATTGAACCGGTGCCTAGAGAAGGTGGCGCGGGGTAAACTGG
GAAAGTGATGTCGTGTACTGGCTCCGCCTTTTTCCCGAGGGTGGGGGAGAACCG
TATATAAGTGCAGTAGTCGCCGTGAACGTTCTTTTTCGCAACGGGTTTGCCGCCA
GAACACAGGTAAGTGCCGTGTGTGGTTCCCGCGGGCCTGGCCTCTTTACGGGTT
ATGGCCCTTGCGTGCCTTGAATTACTTCCACCTGGCTGCAGTACGTGATTCTTGA
TCCCGAGCTTCGGGTTGGAAGTGGGTGGGAGAGTTCGAGGCCTTGCGCTTAAGG
AGCCCCTTCGCCTCGTGCTTGAGTTGAGGCCTGGCCTGGGCGCTGGGGCCGCCG
CGTGCGAATCTGGTGGCACCTTCGCGCCTGTCTCGCTGCTTTCGATAAGTCTCTA
GCCATTTAAAATTTTTGATGACCTGCTGCGACGCTTTTTTTCTGGCAAGATAGTC
TTGTAAATGCGGGCCAAGATCTGCACACTGGTATTTCGGTTTTTGGGGCCGCGG
GCGGCGACGGGGCCCGTGCGTCCCAGCGCACATGTTCGGCGAGGCGGGGCCTGC
GAGCGCGGCCACCGAGAATCGGACGGGGGTAGTCTCAAGCTGGCCGGCCTGCTC
TGGTGCCTGGCCTCGCGCCGCCGTGTATCGCCCCGCCCTGGGCGGCAAGGCTGG
CCCGGTCGGCACCAGTTGCGTGAGCGGAAAGATGGCCGCTTCCCGGCCCTGCTG
CAGGGAGCTCAAAATGGAGGACGCGGCGCTCGGGAGAGCGGGCGGGTGAGTCA
CCCACACAAAGGAAAAGGGCCTTTCCGTCCTCAGCCGTCGCTTCATGTGACTCC
ACGGAGTACCGGGCGCCGTCCAGGCACCTCGATTAGTTCTCGCGCTTTTGGAGT
ACGTCGTCTTTAGGTTGGGGGGAGGGGTTTTATGCGATGGAGTTTCCCCACACTG
AGTGGGTGGAGACTGAAGTTAGGCCAGCTTGGCACTTGATGTAATTCTCCTTGG
AATTTGCCCTTTTTGAGTTTGGATCTTGGTTCATTCTCAAGCCTCAGACAGTGGTT
CAAAGTTTTTTTCTTCCATTTCAGGTGTCGTGAGCTAGCCACCATGTATCCCTAT
GACGTGCCCGATTATGCCGGTGGGGGCGCGCCAACTCAAGACGAATTCACCCAA
CTCTCCCAATCAATCGCCGAGTTCCACACGTACCAACTCGGTAACGGCCGTTGCT
CATCTCTCCTAGCTCAGCGAATCCACGCGCCGCCGGAAACAGTATGGTCCGTGG
TGAGTCGTTTCGATAGGCCACAGATTTACAAACACTTCATCAAAAGCTGTAACG
TGAGTGAAGATTTCGAGATGCGAGTGGGATGCACGCGCGACGTGAACGTGATA
AGTGGATTACCGGCGAATACCTCTCGAGAGAGATTAGATCTGTTGGACGATGAT
CGGAGAGTGACTGGGTTTAGTATAACCGGTGGTGAACATAGGCTGAGGAATTAT
AAATCGGTTACGACGGTTCATAGATTTGAGAAAGAAGAAGAAGAAGAAAGGAT
CTGGACCGTTGTTTTGGAATCTTATGTTGTTGATGTACCGGAAGGTAATTCGGAG
GAAGATACGAGATTGTTTGCTGATACGGTTATTAGATTGAATCTTCAGAAACTTG
CTTCGATCACTGAAGCTATGAACAGCGGAGGAGGAGGTAGCGGAGCGGCCGCT
GGATCCGGATCAGGCTCCNNNNNNNNNNNNNNNNNNNNNNNNNNNNNNNNNN
NNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNN
NNGGATCTGGAGAGGGAAGGGGAAGCCTCCTAACTTGCGGAGATGTCGAGGAG
AATCCTGGCCCAATGGTGAGCAAGGGCGAGGAGGATAACATGGCCATCATCAA
GGAGTTCATGCGCTTCAAGGTGCACATGGAGGGCTCCGTGAACGGCCACGAGTT
CGAGATCGAGGGCGAGGGCGAGGGCCGCCCCTACGAGGGCACCCAGACCGCCA
AGCTGAAGGTGACCAAGGGTGGCCCCCTGCCCTTCGCCTGGGACATCCTGTCCC
CTCAGTTCATGTACGGCTCCAAGGCCTACGTGAAGCACCCCGCCGACATCCCCG
ACTACTTGAAGCTGTCCTTCCCCGAGGGCTTCAAGTGGGAGCGCGTGATGAACT
TCGAGGACGGCGGCGTGGTGACCGTGACCCAGGACTCCTCCCTGCAGGACGGCG
AGTTCATCTACAAGGTGAAGCTGCGCGGCACCAACTTCCCCTCCGACGGCCCCG
TAATGCAGAAGAAGACCATGGGCTGGGAGGCCTCCTCCGAGCGGATGTACCCCG
AGGACGGCGCCCTGAAGGGCGAGATCAAGCAGAGGCTGAAGCTGAAGGACGGC
GGCCACTACGACGCTGAGGTCAAGACCACCTACAAGGCCAAGAAGCCCGTGCA
GCTGCCCGGCGCCTACAACGTCAACATCAAGTTGGACATCACCTCCCACAACGA
GGACTACACCATCGTGGAACAGTACGAACGCGCCGAGGGCCGCCACTCCACCG
GCGGCATGGACGAGCTGTACAAGGGAAGCGGAGCTACTAACTTCAGCCTGCTGA
AGCAGGCTGGTGACGTGGAGGAGAACCCTGGACCTATGACCGAGTACAAGCCC
ACGGTGCGCCTCGCCACCCGCGACGACGTCCCCCGGGCCGTACGCACCCTCGCC
GCCGCGTTCGCCGACTACCCCGCCACGCGCCACACCGTCGACCCGGACCGCCAC
ATCGAGCGGGTCACCGAGCTGCAAGAACTCTTCCTCACGCGCGTCGGGCTCGAC
ATCGGCAAGGTGTGGGTCGCGGACGACGGCGCCGCGGTGGCGGTCTGGACCAC
GCCGGAGAGCGTCGAAGCGGGGGCGGTGTTCGCCGAGATCGGCCCGCGCATGG
CCGAGTTGAGCGGTTCCCGGCTGGCCGCGCAGCAACAGATGGAAGGCCTCCTGG
CGCCGCACCGGCCCAAGGAGCCCGCGTGGTTCCTGGCCACCGTCGGCGTATCGC
CCGACCACCAGGGCAAGGGTCTGGGCAGCGCCGTCGTGCTCCCCGGAGTGGAG
GCGGCCGAGCGCGCCGGGGTGCCCGCCTTCCTGGAGACCTCCGCGCCCCGCAAC
CTCCCCTTCTACGAGCGGCTCGGCTTCACCGTCACCGCCGACGTCGAGGTGCCCG
AAGGACCGCGCACCTGGTGCATGACCCGCAAGCCCGGTGCCTAA
EF1a Promoter: 1-1173
HA Tag: 1186-1212
PYL1 Coding Sequence: 1213-1758
Placeholder Sequence for SEQ ID NOS: 1, 3, 5, 7, 9, 11, 13, 15, 17, 19, and 21: 1828-
2067 (Not all Ns will be present for truncated peptides such as SEQ ID NOS: 3, 5, 7,
17, 19, and 21)
T2A Self-Cleaving Sequence: 2056-2109
mCherry CodingSequence: 2110-2817
P2A Self-Cleaving Peptide Sequence: 2827-2883
Puromycin Resistance Coding Sequence: 2884-3483

1. A degradation targeting agent comprising a ligand moiety and a degradation targeting moiety, wherein the degradation targeting moiety comprises a degradation targeting peptide sequence, wherein the degradation targeting peptide sequence comprises a C3HC4-type zinc-finger domain.

2. The agent of claim 1, wherein the degradation targeting peptide sequence is at least 80%, at least 85%, at least 90%, at least 95%, at least 96%, at least 97%, at least 98%, at least 99%, or 100% identical to any one of SEQ ID NOS: 2, 4, 6, 8, 16, 18, 20, and 22.

3. The agent of claim 1, wherein the degradation targeting sequence comprises at least one, at least two, at least three, or each of:
an isoleucine at a position aligning to position 3 of any one of SEQ ID NOs: 8 and 22;
a phenylalanine at a position aligning to position 20 of any one of SEQ ID NOs: 8 and 22;
a cysteine at a position aligning to position 38 of any one of SEQ ID NOS: 8 and 22; and
a cysteine at a position aligning to position 41 of any one of SEQ ID NOs: 8 and 22.

4. The agent of claim 1, wherein the ligand moiety comprises a peptide, a nucleic acid, or a small molecule.

5. The agent of claim 1, wherein the ligand moiety comprises a peptide.

6. The agent of claim 1, wherein the ligand moiety comprises an antibody or a peptide oligomer.

7. The agent of claim 5, wherein the degradation targeting agent comprises a fusion protein comprising the ligand moiety fused to the degradation targeting peptide.

8. The agent of claim 1, wherein the ligand moiety comprises an aptamer.

9. The agent of claim 1, wherein the ligand moiety binds a protein.

10. The agent of claim 1, wherein the ligand moiety binds an intracellular protein.

11. The agent of claim 1, wherein the ligand moiety binds a nuclear protein.

12. The agent of claim 1, wherein the degradation targeting agent further comprises a nuclear localization signal.

13. A nucleic acid configured to express the degradation targeting agent of claim 1.

14. A method of degrading a target molecule, comprising contacting the target molecule with the degradation targeting agent of claim 1.

15. The method of claim 14, wherein the contacting occurs within a cell.

16. The method of claim 14, wherein the contacting occurs within a nucleus of a cell.

17. The method of claim 14, comprising introducing the degradation targeting agent in a cell comprising the target molecule.

18. The method of claim 14, comprising introducing the degradation targeting agent in a nucleus of a cell comprising the target molecule.

19. The method of claim 14, comprising introducing the degradation targeting agent within the body of a subject.

20. The method of claim 14, comprising administering the degradation targeting agent or a nucleic acid configured to express the degradation targeting agent to a subject.


SEQUENCE LISTING (41 sequences)

SEQ ID NO: 1 - DNA, 240 bp, synthetic construct
  source 1..240: mol_type = other DNA
aagaagctgatcagaaagatgaagctgatcctgagcagcggcagcgacgaggagtgcgccatctgcctggacagcctgaccgtgcccgtgatcacccactgcgcccacgtgttctgcaagccctgcatctgccaggtgatccagaacgagcagccccacgccaagtgccccctgtgcagaaacgacatccacgaggacaacctgctggagtgcccccccgaggagctggccagagacagc

SEQ ID NO: 2 - protein, 80 aa, Homo sapiens
KKLIRKMKLILSSGSDEECAICLDSLTVPVITHCAHVFCKPCICQVIQNEQPHAKCPLCRNDIHEDNLLECPPEELARDS

SEQ ID NO: 3 - DNA, 180 bp, synthetic construct
  source 1..180: mol_type = other DNA
aagaagctgatcagaaagatgaagctgatcctgagcagcggcagcgacgaggagtgcgccatctgcctggacagcctgaccgtgcccgtgatcacccactgcgcccacgtgttctgcaagccctgcatctgccaggtgatccagaacgagcagccccacgccaagtgccccctgtgcaga

SEQ ID NO: 4 - protein, 60 aa, Homo sapiens
KKLIRKMKLILSSGSDEECAICLDSLTVPVITHCAHVFCKPCICQVIQNEQPHAKCPLCR

SEQ ID NO: 5 - DNA, 186 bp, synthetic construct
  source 1..186: mol_type = other DNA
tgcgccatctgcctggacagcctgaccgtgcccgtgatcacccactgcgcccacgtgttctgcaagccctgcatctgccaggtgatccagaacgagcagccccacgccaagtgccccctgtgcagaaacgacatccacgaggacaacctgctggagtgcccccccgaggagctggccagagacagc

SEQ ID NO: 6 - protein, 62 aa, Homo sapiens
CAICLDSLTVPVITHCAHVFCKPCICQVIQNEQPHAKCPLCRNDIHEDNLLECPPEELARDS

SEQ ID NO: 7 - DNA, 126 bp, synthetic construct
  source 1..126: mol_type = other DNA
tgcgccatctgcctggacagcctgaccgtgcccgtgatcacccactgcgcccacgtgttctgcaagccctgcatctgccaggtgatccagaacgagcagccccacgccaagtgccccctgtgcaga

SEQ ID NO: 8 - protein, 42 aa, Homo sapiens
CAICLDSLTVPVITHCAHVFCKPCICQVIQNEQPHAKCPLCR

SEQ ID NO: 9 - DNA, 240 bp, synthetic construct
  source 1..240: mol_type = other DNA
aagaagctgatcagaaagatgaagctgatcctgagcagcggcagcgacgaggagtgcgccgcttgcctggacagcctgaccgtgcccgtgatcacccactgcgcccacgtgttctgcaagccctgcatctgccaggtgatccagaacgagcagccccacgccaagtgccccctgtgcagaaacgacatccacgaggacaacctgctggagtgcccccccgaggagctggccagagacagc

SEQ ID NO: 10 - protein, 80 aa, synthetic construct
KKLIRKMKLILSSGSDEECAACLDSLTVPVITHCAHVFCKPCICQVIQNEQPHAKCPLCRNDIHEDNLLECPPEELARDS

SEQ ID NO: 11 - DNA, 240 bp, synthetic construct
  source 1..240: mol_type = other DNA
aagaagctgatcagaaagatgaagctgatcctgagcagcggcagcgacgaggagtgcgccatctgcctggacagcctgaccgtgcccgtgatcacccactgcgcccacgtggcttgcaagccctgcatctgccaggtgatccagaacgagcagccccacgccaagtgccccctgtgcagaaacgacatccacgaggacaacctgctggagtgcccccccgaggagctggccagagacagc

SEQ ID NO: 12 - protein, 80 aa, synthetic construct
KKLIRKMKLILSSGSDEECAICLDSLTVPVITHCAHVACKPCICQVIQNEQPHAKCPLCRNDIHEDNLLECPPEELARDS

SEQ ID NO: 13 - DNA, 240 bp, synthetic construct
  source 1..240: mol_type = other DNA
aagaagctgatcagaaagatgaagctgatcctgagcagcggcagcgacgaggagtgcgccatctgcctggacagcctgaccgtgcccgtgatcacccactgcgcccacgtgttctgcaagccctgcatctgccaggtgatccagaacgagcagccccacgccaaggctcccctggctagaaacgacatccacgaggacaacctgctggagtgcccccccgaggagctggccagagacagc

SEQ ID NO: 14 - protein, 80 aa, synthetic construct
KKLIRKMKLILSSGSDEECAICLDSLTVPVITHCAHVFCKPCICQVIQNEQPHAKAPLARNDIHEDNLLECPPEELARDS

SEQ ID NO: 15 - DNA, 240 bp, synthetic construct
  source 1..240: mol_type = other DNA
gccagcgccgccagactgaccatgatgtgggaggaggtgacctgccccatctgcctggaccccttcgtggagcccgtgagcatcgagtgcggccacagcttctgccaggagtgcatcagccaggtgggcaagggcggcggcagcgtgtgccccgtgtgcagacagagattcctgctgaagaacctgagacccaacagacagctggccaacatggtgaacaacctgaaggagatcagccag

SEQ ID NO: 16 - protein, 80 aa, Homo sapiens
ASAARLTMMWEEVTCPICLDPFVEPVSIECGHSFCQECISQVGKGGGSVCPVCRQRFLLKNLRPNRQLANMVNNLKEISQ

SEQ ID NO: 17 - DNA, 168 bp, synthetic construct
  source 1..168: mol_type = other DNA
gccagcgccgccagactgaccatgatgtgggaggaggtgacctgccccatctgcctggaccccttcgtggagcccgtgagcatcgagtgcggccacagcttctgccaggagtgcatcagccaggtgggcaagggcggcggcagcgtgtgccccgtgtgcagacagaga

SEQ ID NO: 18 - protein, 56 aa, Homo sapiens
ASAARLTMMWEEVTCPICLDPFVEPVSIECGHSFCQECISQVGKGGGSVCPVCRQR

SEQ ID NO: 19 - DNA, 198 bp, synthetic construct
  source 1..198: mol_type = other DNA
tgccccatctgcctggaccccttcgtggagcccgtgagcatcgagtgcggccacagcttctgccaggagtgcatcagccaggtgggcaagggcggcggcagcgtgtgccccgtgtgcagacagagattcctgctgaagaacctgagacccaacagacagctggccaacatggtgaacaacctgaaggagatcagccag

SEQ ID NO: 20 - protein, 66 aa, Homo sapiens
CPICLDPFVEPVSIECGHSFCQECISQVGKGGGSVCPVCRQRFLLKNLRPNRQLANMVNNLKEISQ

SEQ ID NO: 21 - DNA, 126 bp, synthetic construct
  source 1..126: mol_type = other DNA
tgccccatctgcctggaccccttcgtggagcccgtgagcatcgagtgcggccacagcttctgccaggagtgcatcagccaggtgggcaagggcggcggcagcgtgtgccccgtgtgcagacagaga

SEQ ID NO: 22 - protein, 42 aa, Homo sapiens
CPICLDPFVEPVSIECGHSFCQECISQVGKGGGSVCPVCRQR

SEQ ID NO: 23 - protein, 1009 aa, Homo sapiens
MSWMFKRDPVWKYLQTVQYGVHGNFPRLSYPTFFPRFEFQDVIPPDDFLTSDEEVDSVLFGSLRGHVVGLRYYTGVVNNNEMVALQRDPNNPYDKNAIKVNNVNGNQVGHLKKELAGALAYIMDNKLAQIEGVVPFGANNAFTMPLHMTFWGKEENRKAVSDQLKKHGFKLGPAPKTLGFNLESGWGSGRAGPSYSMPVHAAVQMTTEQLKTEFDKLFEDLKEDDKTHEMEPAEAIETPLLPHQKQALAWMVSRENSKELPPFWEQRNDLYYNTITNFSEKDRPENVHGGILADDMGLGKTLTAIAVILTNFHDGRPLPIERVKKNLLKKEYNVNDDSMKLGGNNTSEKADGLSKDASRCSEQPSISDIKEKSKFRMSELSSSRPKRRKTAVQYIESSDSEEIETSELPQKMKGKLKNVQSETKGRAKAGSSKVIEDVAFACALTSSVPTTKKKMLKKGACAVEGSKKTDVEERPRTTLIICPLSVLSNWIDQFGQHIKSDVHLNFYVYYGPDRIREPALLSKQDIVLTTYNILTHDYGTKGDSPLHSIRWLRVILDEGHAIRNPNAQQTKAVLDLESERRWVLTGTPIQNSLKDLWSLLSFLKLKPFIDREWWHRTIQRPVTMGDEGGLRRLQSLIKNITLRRTKTSKIKGKPVLELPERKVFIQHITLSDEERKIYQSVKNEGRATIGRYFNEGTVLAHYADVLGLLLRLRQICCHTYLLTNAVSSNGPSGNDTPEELRKKLIRKMKLILSSGSDEECAICLDSLTVPVITHCAHVFCKPCICQVIQNEQPHAKCPLCRNDIHEDNLLECPPEELARDSEKKSDMEWTSSSKINALMHALTDLRKKNPNIKSLVVSQFTTFLSLIEIPLKASGFVFTRLDGSMAQKKRVESIQCFQNTEAGSPTIMLLSLKAGGVGLNLSAASRVFLMDPAWNPAAEDQCFDRCHRLGQKQEVIITKFIVKDSVEENMLKIQNKKRELAAGAFGTKKPNADEMKQAKINEIRTLIDL

SEQ ID NO: 24 - protein, 475 aa, Homo sapiens [sequence omitted]

SEQ ID NO: 25 - DNA, 7543 bp, synthetic construct
  source 1..7543: mol_type = other DNA
gtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgcgcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgagctagagcgctgccaccatggacaagaagtacagcatcggcctggacatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgactctagaggtggaggctctggaacgcgtgtgcctttgtatggttttacttcgatttgtggaagaagacctgagatggaagctgctgtttcgactataccaagattccttcaatcttcctctggttcgatgttagatggtcggtttgatcctcaatccgccgctcatttcttcggtgtttacgacggccatggcggttctcaggtagcgaactattgtagagagaggatgcatttggctttggcggaggagatagctaaggagaaaccgatgctctgcgatggtgatacgtggctggagaagtggaagaaagctcttttcaactcgttcctgagagttgactcggagattgagtcagttgcgccggaaacggttgggtcaacgtcggtggttgccgttgttttcccgtcgcacatcttcgtcgctaactgcggtgactccagagccgttctttgccgcggcaaaactgcacttccattatccgttgaccataaaccggatagagaagatgaagctgcgaggattgaagccgcaggagggaaagtgattcagtggaatggagctcgtgttttcggtgttctcgccatgtcgagatccattggcgatagatacttgaaaccatccatcattcctgatccggaagtgacggctgtgaagagagtaaaagaagatgattgtctgattttggcgagtgacggggtttgggatgtaatgacggatgaagaagcgtgtgagatggcaaggaagcggattctcttgtggcacaagaaaaacgcggtggctggggatgcatcgttgctcgcggatgagcggagaaaggaagggaaagatcctgcggcgatgtccgcggctgagtatttgtcaaagctggcgatacagagaggaagcaaagacaacataagtgtggtggtggttgatttgaaggttaagcgacctgccgccacaaagaaggctggacaggctaagaagaagaaagattacaaagacgatgacgataagggatccgagggcagaggaagtctgctaacatgcggtgacgtcgaggagaatcctggcccaccggtcgccaccatggtgagcaagggcgaggagctgttcaccggggtggtgcccatcctggtcgagctggacggcgacgtaaacggccacaagttcagcgtgtctggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggcgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctctacaagggaagcggagctactaacttcagcctgctgaagcaggctggtgacgtggaggagaaccctggacctatggccaagcctttgtctcaagaagaatccaccctcattgaaagagcaacggctacaatcaacagcatccccatctctgaagactacagcgtcgccagcgcagctctctctagcgacggccgcatcttcactggtgtcaatgtatatcattttactgggggaccttgtgcagaactcgtggtgctgggcactgctgctgctgcggcagctggcaacctgacttgtatcgtcgcgatcggaaatgagaacaggggcatcttgagcccctgcggacggtgccgacaggtgcttctcgatctgcatcctgggatcaaagccatagtgaaggacagtgatggacagccgacggcagttgggattcgtgaattgctgccctctggttatgtgtgggagggctaa

SEQ ID NO: 26 - DNA, 4138 bp, synthetic construct
  source 1..4138: mol_type = other DNA
gtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgcgcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgagctagagcgctgccaccatgagcgagctgattaaggagaacatgcacatgaagctgtacatggagggcaccgtggacaaccatcacttcaagtgcacatccgagggcgaaggcaagccctacgagggcacccagaccatgagaatcaaggtggtcgagggcggccctctccccttcgccttcgacatcctggctactagcttcctctacggcagcaagaccttcatcaaccacacccagggcatccccgacttcttcaagcagtccttccctgagggcttcacatgggagagagtcaccacatacgaagacgggggcgtgctgaccgctacccaggacaccagcctccaggacggctgcctcatctacaacgtcaagatcagaggggtgaacttcacatccaacggccctgtgatgcagaagaaaacactcggctgggaggccttcaccgaaacgctgtaccccgctgacggcggcctggaaggcagaaacgacatggccctgaagctcgtgggcgggagccatctgatcgcaaacatcaagaccacatatagatccaagaaacccgctaagaacctcaagatgcctggcgtctactatgtggactacagactggaaagaatcaaggaggccaacaacgagacctacgtcgagcagcacgaggtggcagtggccagatactgcgacctccctagcaaactggggcacaagcttaattctagaggtggaggctctggaacgcgtgtgcctttgtatggttttacttcgatttgtggaagaagacctgagatggaagctgctgtttcgactataccaagattccttcaatcttcctctggttcgatgttagatggtcggtttgatcctcaatccgccgctcatttcttcggtgtttacgacggccatggcggttctcaggtagcgaactattgtagagagaggatgcatttggctttggcggaggagatagctaaggagaaaccgatgctctgcgatggtgatacgtggctggagaagtggaagaaagctcttttcaactcgttcctgagagttgactcggagattgagtcagttgcgccggaaacggttgggtcaacgtcggtggttgccgttgttttcccgtcgcacatcttcgtcgctaactgcggtgactccagagccgttctttgccgcggcaaaactgcacttccattatccgttgaccataaaccggatagagaagatgaagctgcgaggattgaagccgcaggagggaaagtgattcagtggaatggagctcgtgttttcggtgttctcgccatgtcgagatccattggcgatagatacttgaaaccatccatcattcctgatccggaagtgacggctgtgaagagagtaaaagaagatgattgtctgattttggcgagtgacggggtttgggatgtaatgacggatgaagaagcgtgtgagatggcaaggaagcggattctcttgtggcacaagaaaaacgcggtggctggggatgcatcgttgctcgcggatgagcggagaaaggaagggaaagatcctgcggcgatgtccgcggctgagtatttgtcaaagctggcgatacagagaggaagcaaagacaacataagtgtggtggtggttgatttgaaggttaagcgacctgccgccacaaagaaggctggacaggctaagaagaagaaagattacaaagacgatgacgataagggatccgagggcagaggaagtctgctaacatgcggtgacgtcgaggagaatcctggcccaccggtcgccaccatggtgagcaagggcgaggagctgttcaccggggtggtgcccatcctggtcgagctggacggcgacgtaaacggccacaagttcagcgtgtctggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggcgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctctacaagggaagcggagctactaacttcagcctgctgaagcaggctggtgacgtggaggagaaccctggacctatggccaagcctttgtctcaagaagaatccaccctcattgaaagagcaacggctacaatcaacagcatccccatctctgaagactacagcgtcgccagcgcagctctctctagcgacggccgcatcttcactggtgtcaatgtatatcattttactgggggaccttgtgcagaactcgtggtgctgggcactgctgctgctgcggcagctggcaacctgacttgtatcgtcgcgatcggaaatgagaacaggggcatcttgagcccctgcggacggtgccgacaggtgcttctcgatctgcatcctgggatcaaagccatagtgaaggacagtgatggacagccgacggcagttgggattcgtgaattgctgccctctggttatgtgtgggagggctaa

SEQ ID NO: 27 - DNA, 3504 bp, synthetic construct
  source 1..3504: mol_type = other DNA
  misc_difference 1828..2067: note = Up to 114 bases may be deleted
gtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgcgcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgagctagccaccatgtatccctatgacgtgcccgattatgccggtgggggcgcgccaactcaagacgaattcacccaactctcccaatcaatcgccgagttccacacgtaccaactcggtaacggccgttgctcatctctcctagctcagcgaatccacgcgccgccggaaacagtatggtccgtggtgagtcgtttcgataggccacagatttacaaacacttcatcaaaagctgtaacgtgagtgaagatttcgagatgcgagtgggatgcacgcgcgacgtgaacgtgataagtggattaccggcgaatacctctcgagagagattagatctgttggacgatgatcggagagtgactgggtttagtataaccggtggtgaacataggctgaggaattataaatcggttacgacggttcatagatttgagaaagaagaagaagaagaaaggatctggaccgttgttttggaatcttatgttgttgatgtaccggaaggtaattcggaggaagatacgagattgtttgctgatacggttattagattgaatcttcagaaacttgcttcgatcactgaagctatgaacagcggaggaggaggtagcggacctaagaaaaagaggaaggtggcggccgctggatccggatcaggctccnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnggatctggagagggaaggggaagcctcctaacttgcggagatgtcgaggagaatcctggcccaatggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagggaagcggagctactaacttcagcctgctgaagcaggctggtgacgtggaggagaaccctggacctatgaccgagtacaagcccacggtgcgcctcgccacccgcgacgacgtcccccgggccgtacgcaccctcgccgccgcgttcgccgactaccccgccacgcgccacaccgtcgacccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtatcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctaa

SEQ ID NO: 28 - DNA, 3483 bp, synthetic construct
  source 1..3483: mol_type = other DNA
  misc_difference 1807..2046: note = Up to 114 bases may be deleted
gtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgcgcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgagctagccaccatgtatccctatgacgtgcccgattatgccggtgggggcgcgccaactcaagacgaattcacccaactctcccaatcaatcgccgagttccacacgtaccaactcggtaacggccgttgctcatctctcctagctcagcgaatccacgcgccgccggaaacagtatggtccgtggtgagtcgtttcgataggccacagatttacaaacacttcatcaaaagctgtaacgtgagtgaagatttcgagatgcgagtgggatgcacgcgcgacgtgaacgtgataagtggattaccggcgaatacctctcgagagagattagatctgttggacgatgatcggagagtgactgggtttagtataaccggtggtgaacataggctgaggaattataaatcggttacgacggttcatagatttgagaaagaagaagaagaagaaaggatctggaccgttgttttggaatcttatgttgttgatgtaccggaaggtaattcggaggaagatacgagattgtttgctgatacggttattagattgaatcttcagaaacttgcttcgatcactgaagctatgaacagcggaggaggaggtagcggagcggccgctggatccggatcaggctccnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnggatctggagagggaaggggaagcctcctaacttgcggagatgtcgaggagaatcctggcccaatggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagggaagcggagctactaacttcagcctgctgaagcaggctggtgacgtggaggagaaccctggacctatgaccgagtacaagcccacggtgcgcctcgccacccgcgacgacgtcccccgggccgtacgcaccctcgccgccgcgttcgccgactaccccgccacgcgccacaccgtcgacccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtatcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctaa

SEQ ID NO: 29 - protein, 5 aa, synthetic construct
GSGGS

SEQ ID NO: 30 - protein, 4 aa, synthetic construct
GGGS

SEQ ID NO: 31 - protein, 5 aa, synthetic construct
GGGGS

SEQ ID NO: 32 - protein, 5 aa, synthetic construct
GGSGG

SEQ ID NO: 33 - protein, 9 aa, synthetic construct
GGGGSGGGS

SEQ ID NO: 34 - protein, 15 aa, synthetic construct
GGGGSGGGGSGGGGS

SEQ ID NO: 35 - protein, 5 aa, synthetic construct
GPGGP

SEQ ID NO: 36 - protein, 36 aa, synthetic construct
AALVGPGGQGGGGSGGGGSGGGGSGGGGSGGGGSMA

SEQ ID NO: 37 - protein, 15 aa, synthetic construct
EPKSSDKTHTSPPSP

SEQ ID NO: 38 - protein, 12 aa, synthetic construct
GPGGQGTGPGGS

SEQ ID NO: 39 - protein, 7 aa, synthetic construct
PKKKRKV

SEQ ID NO: 40 - protein, 5 aa, synthetic construct
KKKRK

SEQ ID NO: 41 - protein, 30 aa, synthetic construct
  VARIANT 5..13: note = Up to 30 X residues may be inserted
  VARIANT 15..16: note = Up to 2 X residues may be inserted
  VARIANT 17..18: note = 1 X residue may be inserted
  VARIANT 23..26: note = Up to 44 X residues may be inserted
CXXCXXXXXXXXXCXHXXCXXCXXXXCXXC